(19) United States
(12) Patent Application Publication
(10) Pub. No.: US 2003/0208035 A1
(43) Pub. Date: Nov. 6, 2003

(54) Antifungal and/or antibacterial peptides, preparation methods, compositions containing same and methods of treating mammals and/or plants

(75) Inventors: Jean-Luc Dimarcq, Strasbourg (FR); Michele Legrain, Stotzheim (FR); Laure Menin, Cugy (VD) (CH)

Correspondence Address: IP DEPARTMENT OF PIPER RUDNICK LLP, 3400 TWO LOGAN SQUARE, 18TH AND ARCH STREETS, PHILADELPHIA, PA, 19103, US

(73) Assignee: ENTOMED (S.A.), ILLKIRCH (FR)

(21) Appl. No.: 10/340,414

(22) Filed: Jan. 10, 2003

Related U.S. Application Data
(63) Continuation of application No. PCT/FR01/02164, filed on Jul. 5, 2001.

(30) Foreign Application Priority Data
Jul. 13, 2000 (FR) FR 00/09248
Sep. 19, 2000 (FR) FR 00/11949

(51) Int. Cl. A61K 38/16; C07K 14/195

(52) U.S. Cl. 530/324; 514/12

(57) ABSTRACT

The invention concerns peptides derived from helimomicine by substitution of one or several amnio acids, characterised in that the peptides correspond to formula (I) : X₁, X₂, X₃, X₄, X₅, X₆, C₇, X₈, X₉, X₁₀, X₁₁, X₁₂, X₁₃, X₁₄, X₁₅, X₁₆, X₁₇, C₁₈, X₁₉, X₂₀, X₂₁, C₂₂, X₂₃, X 24, X25, X₂₆, X₂₇, X₂₈, X₂₉, X₃₀, X₃₁, X₃₂, X₃₃, X₃₄, X₃₅, X₃₆, X₃₇, X₃₈, X₃₉, C₄₀, X₄₁, C₄₂, X₄₃, X₄₄ wherein X₁, X₁₇, X₂₁, X₄₃ are amino acids; X₁₆, X₄₄ are small polar amino acids; X₁₉ is a large polar amino acid; X₃₆ is a small or lightly hydophobic amino acid; X₃₈ is a lightly hydrophobic or small amino acid; the substitutions being such that: at least one of X₁, X₁₇, X₂₁, X₄₃ is a basic or polar, advantageously large polar amino acid, and/or at least one of the amnio acids X₁₆, X₄₄ is a basic amino acid or a large polar amino acid, and/or X₁₉ is a basic amino acid, and/or at least one of the amino acids X₃₆, X₃₈ is a strongly hydrophobic amino acid. The invention also concerns antifungal and/or antibacterial compositions comprising at least one of the peptides.

### RELATED APPLICATION

This is a continuation of International Application No. PCT/FR01/02164 filed Jul. 5, 2001, which claims benefit from French Patent Application No. 00/09248 filed Jul. 13, 2000 and French Patent Application No. 00/11949 filed Sep. 19, 2000.

### FIELD OF THE INVENTION

This invention relates to new peptides having antibacterial and antifungal properties. The invention also concerns the preparation of these peptides and compositions containing the same which may be used in agriculture and for human or animal therapy.

### BACKGROUND

In the prior art, numerous substances of natural origin are described, in particular, peptides having antimicrobial properties and, more particularly, bactericides and fungicides. Such peptides may be used to treat fungal diseases both in plants and in man (De Lucca et al., 1999, Antimicrob. Agents Chemother. 43, 1-11). In human health, it can be recalled that the frequency of opportunistic fungal infections has risen sharply in recent years. Invasive mycoses are very serious infections caused by fungi found in nature and which become pathogenic in immunocompromised persons. Immunosuppression may be the result of various causes: corticotherapy, chemotherapy, transplants, HIV infection. Opportunistic fungal infections currently account for a high mortality rate in man. They may be caused by yeasts, mainly of Candida type, or filamentous fungi, chiefly of Aspergillus type. In immunosuppressed patients, failure of antifungal treatment is frequently observed on account of its toxicity, for example, treatment with Amphotericin B, or the onset of resistant fungi, for example resistance of Candida albicans to nitrogen derivatives. It is, therefore, vital to develop new antifungal medicinal products derived from innovative molecules

The production of antimicrobial peptides, in a large variety of animal and plant species, represents an essential mechanism in immunity defence against infections. Insects, in particular, show very effective resistance against bacteria and fungi. This response is largely attributable to the rapid synthesis of several families of wide spectrum antimicrobial peptides (Bulet et al. (1999) Dev. Comp. Immunol. 23, 329-344). This synthesis is induced by a septic injury or injection of a low dose of bacteria (Hoffmal et al. (1999) Science 284, 1313-1318). To date, the antimicrobial peptides of insects have especially been characterized from insects undergoing complete metamorphosis during their development, Diptera, Lepidoptera and Coleoptera, for example. Among the anti-microbial peptides induced in these insects, a distinction may be made between the four following groups:

Cationic peptides of 4 kDa, forming two amphipathic α-helixes. This group particularly includes cecropins.

Cationic peptides rich in proline, having a size of between 2 kDa and 4 kDa which may be glycosylated, such as drosocine, pyrrhocoricine and the lebocines, for example, or non-glycosylated such as the apidaecines and metalnikowines.

Several separate polypeptides with a molecular weight of 8 to 27 kDa, cationic for the most part and frequently rich in glycine residues such as attacines, II sarcotoxins, diptericines and coleoptericine.

Peptides containing intramolecular disulfide bridges. This group contains insect defensines (4 kDa, 3 disulfide bridges), drosomycin (4 kDa, 4 disulfide bridges) and thanatine (2 kDa, 1 disulfide bridge).

### SUMMARY OF THE INVENTION

This invention relates to a peptide derived from heliomycin by substituting one or more amino acids, including peptides meeting formula (I):

X₁ X₂ X₃ X₄ X₅ X₆ C₇ X₈ X₉ X₁₀ X₁₁ X₁₂ X₁₃ X₁₄ X₁₅ X₁₆ X₁₇ C₁₈ X₁₉ X₂₀ X₂₁ C₂₂ X₂₃ X₂₄ X₂₅ X₂₆ X₂₇ X₂₈ X₂₉ X₃₀ X₃₁ C₃₂ X₃₃ X₃₄ X₃₅ X₃₆ X₃₇ X₃₈ X₃₉ C₄₀ X₄₁ C₄₂ X₄₃ X₄₄ (I)

in which:

X₁, X₁₇, X₂₁, X₄₂, are acidic amino acids,

X₁₆, X₄₄ are small polar amino acids,

X₁₉ is a large polar amino acid,

X₃₆ is a small or scarcely hydrophobic amino acid,

X₃₈ is a scarcely hydrophobic or small amino acid, the substitutions being such that:

at least one of X₁, X₁₇, X₂₁, X₄₃, is a basic or polar, advantageously a large polar, amino acid and/or

at least one of the amino acids X₁₆, X₄₄ is a basic amino acid or a large polar amino acid, and/or

X₁₉ is a basic amino acid, and/or

at least one of the amino acids X_{36,} X₃₈ is a strongly hydrophobic amino acid,

and in which other amino acids (X) have the following meanings:

X₁₃, X₃₇, X₃₉ represent large polar amino acids,

X₅, X₁₅, X₃₄, represent small polar amino acids,

X₂, X₂₃, X₂₄, X₂₅, X₂₈, X₃₁, represent basic amino acids,

X₃, X₄, X₈, X₁₂, represent hydrophobic amino acids,

X₉, X₁₆, X₂₇, X₃₅, X₄₁ represent aromatic hydrophobic amino acids,

X₅, X₁₀, X₁₁, X₂₀, X_{26,} X₂₉, X₃₀, X₃₃, represent small amino acids,

C_{7,} C_{18,} C_{22,} C_{32,} C_{40,} C_{42,} represent cysteines.

Also, this invention relates to an antifungal and/or antibacterial composition comprising a therapeutically effective amount of at least one peptide as described above, and a pharmaceutically acceptable vehicle.

### BRIEF DESCRIPTION OF THE DRAWINGS

Other advantages and characteristics of the invention will become apparent on reading the following examples concerning the preparation of the Ard1 peptide and analogues of heliomycin and Ard1, and their antifungal activity, with reference to the appended drawings in which:

FIG. 1 shows the hydrophobicity profile of the Heliomycin peptide using Kyte and Doolittle's method (1982, J. Mol. Biol., 157, 105-132);

FIG. 2 shows the activities (survival rate relative to post-infection days) of the peptides Heliomycin and Ard1 in the infection model with disseminated Candida albicans;

FIG. 3 shows the activities (morbidity scores relative to post-infection days) of the Heliomycin and Ard1 peptides in the infection model with disseminated Candida albicans;

FIG. 4 shows the activities (survival rate in relation to post-infection days) of the peptides pEM24, pEM30, pEM31 and pEM35 in the infection model with disseminated Candida albicans;

FIG. 5 shows the activities (morbidity scores in relation to post-infection days) of the peptides pEM24, pEM30, pEM31 and pEM35 in the disseminated Candida albicans infection model;

FIG. 6 shows the activities (survival rate in relation to post-infection days) of the peptides pEM31, pEM35, pEM46 and pEM51 in the disseminated Candida albicans infection model;

FIG. 7 shows the activities (morbidity scores in relation to post-infection days) of the peptides pEM31, pEM35, pEM46 an dpEM51 in the disseminated Candida albicans infection model;

FIG. 8 shows the activities (survival rate in relation to post-infection days) of the pEM35 peptide in the disseminated Candida albicans infection model;

FIG. 9 shows the activities (survival rate relative to post-infection days) of the pEM35 and pEM51 peptides in the disseminated Scedosporium inflatum infection model;

FIG. 10 shows the activities (morbidity rate relative to post-infection days) of the pEM35 and pEM51 peptides in the disseminated Scedosporium inflatum infection model;

FIG. 11 shows weight changes in relation to time in healthy mice treated with the pEM51 peptide;

FIG. 12 shows weight changes in relation to time in healthy mice treated with the pEM35 and pEM51 peptides;

FIG. 13 shows the fungicidal kinetics of the pEM35 and pEM51 peptides against Candida albicans IHEM 8060.

### DETAILED DESCRIPTION

This invention takes particular interest in peptides of three-dimensional structure of the type containing one α-helix and one antiparallel β strand joined by three disulfide bridges, also called a CSαβ structure. These peptides have antifungal activity that is useful for testing infections in man, animals and in plants. The invention particularly concerns heliomycin which is a peptide isolated from the haemolymph of the Lepidoptera Heliothis virescens. The sequence and properties of heliomycin are described in published PCT N^{o} WO 9953053.

In the peptide sequences listed below, the amino acids are represented by their one-letter code, but they could also be represented by their three-letter code in accordance with the following nomenclature:

| | | |
|---|---|---|
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Heliomycin is an amphiphilic peptide having a three-dimension structure of CSαβ type. The amino acid sequence of heliomycin given in the list of sequences under number SEQ ID No : 1 is the following:

| | |
|---|---|
| 1 10 20 30 | |
| D K L I G S C V W G A V Y T S D C N G E C K R R G Y K G G | (SEQ ID NO: 1) |
| 40 | |
| H C G S F A N V N C W C E T | |

We have now, from the haemolymph of immunized larvae of the Lepidoptera Archeoprepona demophoon, isolated a homologue of heliomycin. This peptide, called Ard1, was characterized by sequencing and mass measurement. The amino acid sequence of Ard1 is shown in the sequence list under number SEQ ID NO : 2

| | |
|---|---|
| 1 10 20 30 | |
| D K L I G S C V W G A V N Y T S N C N A E C K R R G Y K G G | (SEQ ID NO: 2) |
| 40 | |
| H C G S F A N V N C W C E T | |

The sequence of Ard1 differs from that of heliomycin at 2 positions: an aspartic acid (Asp) at position 17 in heliomycin is replaced by an asparagine (Asn), and a glycine (Gly) at position 20 is replaced by an alanine (Ala). The corresponding codons were modified in the expression vector pSEA2 of heliomycin and the Ard1 peptide was produced and secreted by the yeast S. cerevisiae.

pSEA2 is a yeast expression vector carrying the MFα1 promoter and the pre sequence of BGL2 and pro sequence of MFα1 permitting secretion of the peptide in the culture medium (Lamberty et al., 1999, J. Biol. Chem., 274, 9320-9326).

After HPLC purification, the antifungal activity (anti-Candida albicans and anti-Aspergillus fumigatus activity) of Ard1 were compared with that of heliomycin. The anti-Candida albicans activity of Ard1 is 4 to 8 times greater than that of heliomycin. The anti-Aspergillus fumigatus activity of Ard1 is 2 times greater than that of heliomycin.

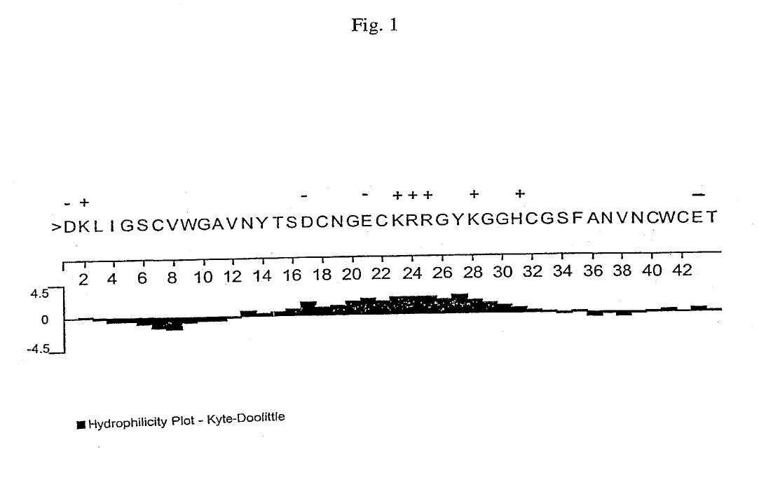

We analysed the charge and hydrophobicity of heliomycin and of the Ard1 peptide. The hydrophobicity profile shown in appended FIG. 1 was made following the method of Kyte and Doolittle (1982, J. Mol. Biol., 157, 105-132).

Heliomycin and its homoloque Ard1 have two regions of rather hydrophobic nature separated by a region that is more hydrophilic. The N and C end regions are rather hydrophilic. Also, the central region that is of hydrophilic nature has a positive net charge. FIG. 1 shows the charge of the amino acids in the heliomycin sequence.

The replacement of aspartic acid in heliomycin by asparagine (position 17) in the natural homologue Ard1 increases the cationic nature of the peptide (+1 relative to heliomycin). Other mutations intended to increase the positive charge and hydrophobicity were made in heliomycin and its homologue Ard1 by PCR-generated directed mutagenesis or by cloning synthetic fragments.

Research conducted under the scope of this invention, therefore, consisted of making mutations particularly in the hydrophobic, charged regions to increase the charge and/or hydrophobicity of the peptides without modifying or by improving their amphophilic nature, and in this manner to produce peptides having improved antifungal and/or antibiotic properties relative to heliomycin.

This purpose is achieved by means of a peptide derived from heliomycin having the formula SEQ ID NO 1:

DKLIGSCVWGAVNYTSDCNGECKRRGYKGGHCGSFANVNCWCET

by substitution of one or more amino acids. The peptides of the invention meet formula (I) in which “X” represents an amino acid:

X₁ X₂ X₃ X₄ X₅ X₆ C₇ X₈ X₉ X₁₀ X₁₁ X₁₂ X₁₃ X₁₄ X₁₅ X₁₆ X₁₇ C₁₈ X₁₉ X₂₀ X₂₁ C₂₂ X₂₃ X₂₄ X₂₅ X₂₆ X₂₇ X₂₈ X₂₉ X₃₀ X₃₁ C₃₂ X₃₃ X₃₄ X₃₅ X₃₆ X₃₇ X₃₈ X₃₉ C₄₀ X₄₁ C₄₂ X₄₃ X₄₄ (I)

in which:

X₁, X₁₇, X₂₁, X₄₃ are acidic amino acids,

X₁₆, X₄₄ are small polar amino acids,

X₁₉ is a large polar amino acid,

X₃₆ is a small or weakly hydrophobic amino acid,

X₃₈ is a scarcely hydrophobic or small amino acid, said substitutions being such that:

at least one of X₁, X₁₇, X₂₁, X₄₃ is basic or polar, advantageously a large polar, amino acid and/or

at least one of amino acids X₁₆, X₄₄ is a basic amino acid or a large polar amino acid, and/or

X₁₉ is a basic amino acid, and/or

at least one of amino acids X_{36,} X₃₈ is a strongly hydrophobic amino acid,

and in which, the other amino acids (X) have the following meanings:

X₁₃, X₃₇, X₃₉ represent large polar amino acids,

X₆, X₁₅, X₃₆ represent small polar amino acids,

X₂, X₂₃, X₂₄, X₂₅, X₂₈, X₃₁ represent basic amino acids,

X₃, X₄, X₈, X₁₂ represent hydrophobic amino acids,

X₉, X₁₄, X₂₇, X₃₅, X₄₁ represent aromatic hydrophobic amino acids,

X₅, X₁₀, X₁₁, X₂₀, X₂₅, X₂₉, X₃₀, X₃₃ represent small amino acids,

C_{7,} C_{18,} C_{22,} C_{32,} C_{40,} C₄₂ represent cysteines.

Therefore, in the peptides of the invention of formula (I), when:

all or part of X₁, X₁₇, X₂₁, X₄₃ is not basic or polar, advantageously a large polar, amino acid it is or they are an acidic amino acid or acids,

all or part of X₁₆, X₄₄ is not basic or large polar amino acid, it is or they are a small polar amino acid,

X₁₉ is not a basic amino acid, it is a large polar amino acid,

X₃₆ is not a strongly hydrophobic amino acid, it is a small or scarcely hydrophobic amino acid,

X₃₈ is not a strongly hydrophobic amino acid, it is a scarcely hydrophobic or small acid.

The peptides of the invention have the CSαβ structure of heliomycin since the substitutions do not concern cysteines C₇, C_{18,} C_{22,} C_{32,} C_{40,} C₄₂.

One first preferred group of peptides according to the invention is the group in which at least one of X₁, X₁₇, X₄₃ is a basic or polar, advantageously a large polar, amino acid, and X₂₁ is an acidic amino acid able to set up ion bonds with at least one of X₂₃, X₂₄ and X₂₅ which are basic amino acids. These bonds are able to take part in the stabilisation of the CSαβ structure of the peptides of the invention.

A second preferred group of peptides according to the invention is the group in which at least one of X₃₆ and X₃₈ is a non-aromatic strongly hydrophobic amino acid.

A third preferred group of peptides according to the invention is the group in which X₁₇ is asparagine or arginine, X₄₃ is glutamic acid and in which:

X₃₆ is leucine or isoleucine, and/or

X₁₉ is arginine, and/or

X₁₆ is arginine.

A fourth preferred group of peptides according to the invention is the group in which X₁₇ is aspartic acid, X₄₃ is glutamic acid and in which:

X₃₆ is leucine or isoleucine, and/or

X₁₉ is arginine, and/or

X₁₆ is arginine.

A fifth preferred group of peptides according to the invention is the group in which X₄₃ is glutamine, X₁₇ is asparagines, and in which:

X₃₆ is leucine or isoleucine, and/or

X₁₉ is arginine.

A sixth preferred group of peptides according to the invention is the group in which X₄₃ is glutamine and X₁₇ is aspartic acid.

A seventh preferred group of peptides according to the invention is the group in which X₄₃ is glutamine, X₁₇ is aspartic acid and in which:

X₁ is asparagine, and/or

X₃₆ is leucine or isoleucine.

The following meanings are given:

Basic amino acids: arginine, lysine or histidine.

Hydrophobic amino acids:

non-aromatic: methionine, valine, leucine, isoleucine, on the understanding that leucine and isoleucine are strongly hydrophobic amino acids, and methionine and valine are scarcely hydrophobic amino acids,

aromatic: phenylalanine, tyrosine or tryptophan which are strongly hydrophobic amino acids,

acidic amino acids: aspartic acid or glutamic acid,

large polar amino acids, glutamine or asparagine,

small polar amino acids: serine or threonine,

polar amino acids: small and large polar amino acids,

small amino acids: glycine or alanine.

The peptides of the invention may be prepared by chemical synthesis or genetic engineering using techniques well known to persons skilled in the art.

Three types of mutations in particular were generated:

acidic amino acids were replaced by polar amino acids, such as Asp1 mutations to Asn, Asp17 to Asn, Glu43 to Gln, and

polar, preferably large polar, amino acids were replaced by basic amino acids, such as the mutations of Asn13 to Arg, Ser16 to Arg, Asn17 to Arg (Ard1), Asn19 to Arg, Thr44 to Arg.

mutations tending to increase hydrophobicity were also generated, such as the mutations Gly10 to Leu, Ala36 to Leu or Ile and Val38 to Ile.

Preferred peptides derived from heliomycin according to the invention have the following amino acid sequences:

| | |
|---|---|
| Helio: | DKLIGSCVWGAVNYTSDCNGECKRRGYKGGHCGSFANVNCWCET |
| Ard1: | DKLIGSCVWGAVNYTSNCNAECKRRGYKGGHCGSFANVNCWCET |
| pEM37: | NKLIGSCVWGAVNYTSDCNGECKRRGYKGGHCGSFANVNCWCET |
| pEM38: | DKLIGTCVWGAVNYTSDCNGECKRRGYKGGHCGSFANVNCWCET |
| pEM43: | DKLIGSCVWGAVNYTTDCNGECKRRGYKGGHCGSFANVNCWCET |
| pEM42: | DKLIGSCVWGAVNYTRDCNGECKRRGYKGGHCGSFANVNCWCET |
| pEM44: | DKLIGSCVWGAVNYTSDCRGECKRRGYKGGHCGSFANVNCWCET |
| pEM22: | DKLIGSCVWGAVNYTSDCNGECKRRGYKGGHCGSFINVNCWCET |
| pEM23: | DKLIGSCVWGAVNYTSDCNGECKRRGYKGGHCGSFANINCWCET |
| pEM25: | DKLIGSCVWGAVNYTSDCNGECKRRGYKGGHCGSFLNVNCWCET |
| pEM24: | DKLIGSCVWGAVNYTSDCNGECKRRGYKGGHCGSFLNINCWCET |
| pEM7: | DKLIGSCVWGAVNYTSDCNGECKRRGYKGGHCGSFANVNCWCER |
| pEM21: | DKLIGSCVWGAVNYTSDCNGECKRRGYKGGHCGSFANVNCWCQT |
| pEM39: | NKLIGSCVWGAVNYTSDCNGECKRRGYKGGHCGSFANVNCWCQT |
| pEM61: | NKLIGSCVWGAVNYTSDCNGECKRRGYKGGHCGSFLNVNCWCQT |
| pEM62: | NKLIGSCVWGAVNYTSDCNGECKRRGYKGGHCGSFINVNCWCQT |

Preferred peptides derived from Ard1 according to the invention have the following amino acid sequences:

Ard1: DKLIGSCVWGAVNYTSNCNAECKRRGYKGGUCGSFANVNCWCET

pEM40: NKLIGSCVWGAVNYTSNCLWSCKRRGYKGGHCGSFANVNCWCET

| | |
|---|---|
| Ard1: | DKLIGSCVWGAVNYTSNCNAECKRRGYKGGHCGSFANVNCWCET |
| pEM40: | NKLIGSCVWGAVNYTSNCNAECKRRGYKGGHCGSFANVNCWCET |
| pEM50: | DKLIGSCVWGAVNYTRNCNAECKRRGYKGGHCGSFANVNCWCET |
| pEM56: | DKLIGSCVWLAVNYTSNCNAECKRRGYKGGHCGSFANVNCWCET |
| pEM52: | DKLIGSCVWGAVNYTSRCNAECKRRGYKGGHCGSFANVNCWCET |
| pEM51: | DKLIGSCVWGAVNYTSNCRAECKRRGYKGGHCGSFANVNCWCET |
| pEM32: | DKLIGSCVWGAVNYTSNCNAECKRRGYKGGHCGSFINVNCWCET |
| pEM33: | DKLIGSCVWGAVNYTSNCNAECKRRGYKGGHCGSFANINCWCET |
| pEM34: | DKLIGSCVWGAVNYTSNCNAECKRRGYKGGHCGSFLNINCWCET |
| pEM35: | DKLIGSCVWGAVNYTSNCNAECKRRGYKGGHCGSFLNVNCWCET |
| pEM31: | DKLIGSCVWGAVNYTSNCNAECKRRGYKGGHCGSFANVNCWCQT |
| pEM30: | DKLIGSCVWGAVNYTSNCNAECKRRGYKGGHCGSFANVNCWCER |
| pEM46: | DKLIGSCVWGAVNYTSNCNAECKRRGYKGGHCGSFLNVNCWCQT |
| pEM47: | DKLIGSCVWGAVNYTSNCNAECKRRGYKGGHCGSFINVNCWCQT |
| pEM48: | DKLIGSCVWGAVNYTSNCNAECKRRGYKGGHCGSFLNVNCWCER |
| pEM49: | DKLIGSCVWGAVNYTSNCNAECKRRGYKGGHCGSFINVNCWCER |
| pEM54: | DKLIGSCVWLAVNYTSNCNAECKRRGYKGGHCGSFLNVNCWCET |
| pEW57: | DKLIGSCVWLAVNYTSNCNAECKRRGYKGGHCGSFANVNCWCQT |
| pEM55: | DKLIGSCVWLAVNYTSNCNAECKRRGYKGGHCGSFLNVNCWCQT |

The invention also concerns functional equivalents of the above peptides. These may, for example, be fragments of the above peptides or modifications resulting from post-translation processes such as glyco-sylation or chemical modifications such as amidation, acetylation, acylation, coupling with lipids or sugars, coupling with nucleotides and the like.

The functional equivalents also comprise peptides of the invention in which one or more amino acids are enantiomers, diasteroisomers, natural amino acids of D conformation, rare amino acids particularly hydroxy-proline, methyllysine, dimethyllysine, and synthetic amino acids particularly ornithine, norleucine, cyclo-hexylalanine and omega-aminoacids. The invention also covers retropeptides and retro-inversopeptides.

The peptides of formula (I) may also, at either one of their N- or C- terminal ends, comprise one or more amino acids which do not interfere with the structure of formula (I) The invention evidently covers peptides having a three-dimensional structure of the type containing one α-helix and one antiparallel β strand joined by three disulfide bridges, such as heliomycin.

Table 1 below gives the mutations made on the amino acids at positions 1, 6, 13, 16, 19, 36, 38, 43 and 44 of heliomycin, and the antifungal activity of the peptides obtained on C. albicans (C.a.) and A. fumigatus (A.f.).

TABLE 1

| Position | | | | | | | | |
|---|---|---|---|---|---|---|---|---|
| 1 | 6 | 13 | 16 | 19 | 36 | 38 | 43 | 44 |

| | |
|---|---|
| Heliomycin | Activity⁺ |

| Mutants | D | S | N | S | N | A | V | E | T | C.a. | A.f. |
|---|---|---|---|---|---|---|---|---|---|---|---|
| pEM37 | N | | | | | | | | | 2 | — |
| pEM38 | | T | | | | | | | | 1 | 1 |
| pEM45 | | | R | | | | | | | 0.5 | <<<1 |
| pEM43 | | | | T | | | | | | 1 | 2 |
| pEM42 | | | | R | | | | | | 8 | 1 |
| pEM44 | | | | | R | | | | | 8 | 2 |
| pEM22 | | | | | | I | | | | 1-2 | 4-8 |
| pEM23 | | | | | | | I | | | 1 | 2 |
| pEM25 | | | | | | L | | | | 10 | 6 |
| pEM24 | | | | | | L | I | | | 4 | 8 |
| pEM7 | | | | | | | | | R | 4-8 | 1 |
| pEM21 | | | | | | | | Q | | 2 | 10-20 |
| pEM39 | N | | | | | | | Q | | 2 | 4-8 |
| pEM61 | N | | | | | | L | Q | | 5-10 | 3-6 |
| pEM62 | N | | | | | | I | Q | | 3-6 | 2-4 |

⁺relative activity in relation to heliomycin

Table 2 below gives the mutations made on the amino acids at positions 1, 10, 16, 17, 19, 36, 38, 43 and 44 of the Ard1 peptide, and the antifungal activity of the peptides obtained on C. albicans (C.a.) and A. fumigatus (A.f.).

TABLE 2

| Position | | | | | | | | |
|---|---|---|---|---|---|---|---|---|
| 1 | 10 | 16 | 17 | 19 | 36 | 38 | 43 | 44 |

| | |
|---|---|
| Ard1 | Activity⁺ |

| Mutants | D | G | S | N | N | A | V | E | T | C.a. | A.f. |
|---|---|---|---|---|---|---|---|---|---|---|---|
| pEM40 | N | | | | | | | | | 2 | 1 |
| pEM50 | | | R | | | | | | | 1-2 | 1 |
| pEM56 | | L | | | | | | | | 1-2 | 0.5 |
| pEM52 | | | | R | | | | | | 1-2 | 0.5 |
| pEM51 | | | | | R | | | | | 2-4 | 1 |
| pEM32 | | | | | | I | | | | 1 | 2 |
| pEM33 | | | | | | | I | | | 1 | 1 |
| pEM34 | | | | | | L | I | | | 4 | 4 |
| pEM35 | | | | | | L | | | | 4 | 2-4 |
| pEM31 | | | | | | | | Q | | 2 | 4-8 |
| pEM30 | | | | | | | | | R | 4 | 0.5-1 |
| PEM46 | | | | | | L | | Q | | 4-8 | 6-8 |
| pEM47 | | | | | | I | | Q | | 2-4 | 8 |
| pEM48 | | | | | | L | | | R | 6-12 | 1-2 |
| pEM49 | | | | | | I | | | R | 3 | 1-2 |
| pEM54 | | L | | | | L | | | | 1 | 1 |
| pEM57 | | L | | | | | | Q | | 1 | 8 |
| pEM55 | | L | | | | L | | Q | | 1-2 | 2-7 |

⁺relative activity in relation to Ard1

The different mutants were produced in S. cerevisiae yeast, HPLC purified and their antifungal activity (C. albicans and A. fumigatus) was compared with that of heliomycin or the Ard1 peptide.

Tables 1 and 2 above show a gain in activity on at least one of the two tested fungi for all mutants with increased positive charge with the exception of the Asn 13 mutant to Arg (pEM45). The other mutants are all localized in hydrophilic regions. The majority of mutants have increased activity on C. albicans (Ser16 to Arg, Asn17 to Arg (Ard1), Asn19 to Arg, Thr44 to Arg). One single mutation (Glu43 to Gln) provided a significantly substantial gain in activity on A. fumigatus.

Concerning the mutations with increase in hydrophobicity, the change of Ala36 to Leu (pEM35) gives the best gain in activity on C. albicans and A. fumigatus. The mutations Gly10 to Leu and Val38 to Ile have no significant effect on the antifungal activity of heliomycin and Ard1.

The mutants with the most active increase in hydrophobicity were associated with the mutants with increased charge. Cumulative effects were hence observed.

The invention also relates to the use of the above peptides to prevent or treat a fungal and/or bacterial infection both in man and animal and in plants. A subject of the invention, is therefore, a composition, more particularly an antifungal and/or antibacterial pharmaceutical composition, containing as an active ingredient at least one peptide as previously defined, advantageously associated in said composition with an acceptable vehicle.

The vehicle is chosen in relation to the type of application of the composition for pharmaceutical or agronomical purposes.

The invention particularly concerns pharmaceutical applications in man and animal of these peptides and compositions containing the same, but it also concerns agronomical applications. The peptides of the invention can be used to make plants resistant to disease, fungal and bacterial disease in particular. One first embodiment of this agronomical application consists of applying to plants an efficient quantity of peptides or composition containing the same. A second embodiment of this agronomical application consists of transforming plant cells or plants with a nucleic acid sequence able to express the peptide of the invention to impart disease resistance to the plants.

### EXAMPLE 1

Isolation of Ard1 from Haemolymph taken from Immunized Larvae of the A. demophoon Lepidoptera.

1) Induced Biological Synthesis of an Antifungal Substance in the Haemolymph of A. demophoon.

Stage-4 mature larvae of the A. demophoon Lepidoptera were immunized with two injections of 20 μl PBS solution containing gram-positive bacteria (M. luteus and S. aureus), gram-negative bacteria (P. aeruginosa), spores of filamentous fungi (A. fumigatus) and yeasts (C. albicans). The bacteria were prepared from cultures made in Luria-Bertani medium for 12 hours at 37° C. The yeasts were prepared from cultures made in Sabouraud medium for 12 hours at 30° C. The spores of A. fumigatus were taken from stock frozen at −90° C. The animals infected in this manner were kept for 24 hours on their host plant, in a ventilated area. Before removing the haemolymph the larvae were cooled on ice.

2) Preparation of the Plasma

The haemolymph (approximately 160 μl per larva, for a total number of 81 specimens) was collected by excising an abdominal appendix and placed in 1.5 ml polypropylene micro-centrifugation tubes cooled on ice and containing aprotinine as protease inhibitor (20 μg/ml final concentration) and phenylthiourea as melanization inhibitor (final concentration of 40 μM). The haemolymph (13 ml) collected from the immunized larvae was centrifuged at 8000 rpm for 1 min at 4° C. to remove the hemocytes. The supernatant from centrifugation was centrifuged at 12000 rpm. The haemolymph free of its blood cells was stored at −80° C. until use.

3) Plasma Acidification

After fast thawing, the plasma of A. demophoon was acidified to pH3 with a 1% (volume/volume) solution of trifluoroacetic acid containing aprotinine (20 μg/ml final concentration)) and phenylthiourea (final concentration of 40 μM). Extraction of the peptide under acid conditions was performed for 30 min under slight shaking over an iced water bath. The extract obtained was then centrifuged at 4° C. for 30 min at 10000 g.

4) Peptide Purification

a) Prepurification by Solid Phase Extraction

A quantity of extract equivalent to 5 ml of haemolymph was deposited on a 2 g reverse phase carrier, such as commercially available in cartridge form (Sep-Pak™ C18, Waters associates, equilibrated with acidified water (0.05% TFA). The hydrophilic molecules were removed by simple washing with acidified water. Elution of the peptide was made using a 60% solution of acetonitrile prepared in the 0.05% TFA. The fraction eluted with 60% acetonitrile was vacuum dried to remove the acetonitrile and TFA and it was then reconstituted in sterile acidified water (0.05% TFA) before undergoing the first purification step.

b) High Performance Liquid Chromatography (HPLC) Purification on Reverse Phase Column.

step one: the fraction containing the peptide was analysed by reverse phase chromatography on an Aquapore RP-300 C₈ preparation column (Brownlee™, 220×10 mm, 300 A), elution was performed on an acetonitrile gradient in 0.05% TFA, from 2% to 10% in 5 minutes, then from 10 to 25% in 30 minutes, then 25% to 35% in 40 minutes, then 35% to 60% in 50 minutes, for a total duration of 125 minutes at a constant rate of 2.5 ml/min. The fractions were collected manually following absorbency variation at 225 nm. The collected fractions were vacuum dried, reconstituted with ultrapure water and analysed for antifungal activity using the test described below.

step two: the antifungal fraction eluted at 27% acetonitrile corresponding to the peptide was analysed on an Aquapore RP-300 C₈ reverse phase analytical column (Brownlee™, 220×4.6 mm, 300 A), using a diphase linear gradient of acetonitrile of 2% to 23% in 5 min and 23 to 31% in 50 min in 0.05% TFA at a constant rate of 0.8 ml/min. The fractions were collected manually following absorbency variation at 225 nm. The collected fractions were vacuum dried, reconstituted with ultrapure water and their antifungal activity analysed under the conditions described below.

step three: the antifungal fraction containing the peptide was purified to homogeneity on a reverse phase Narrowbore Delta-Pak™ HPI C₁₈ column (Waters Associates, 150×2 mm) using a diphase linear gradient of acetonitrile from 2% to 22% in 5 min and from 22 to 30% in 50 min in 0.05% TFA at a constant rate of 0.25 ml/min at a controlled temperature of 30° C. The fractions were collected manually following absorbency variation at 225 nm. The collected fractions were vacuum dried, reconstituted with filtered ultrapure water and their antifungal activity analysed.

### EXAMPLE 2

Structural Characterization of the Ard1 Peptide.

1) Purity Checking by MALDI-TOF Mass Spectrometry (Matrix Assisted Laser Desorption Ionization—Time of Flight).

Purity checking was performed on MALDI-TOF Bruker Biflex mass spectrometry equipment (Bremen, Germany) in positive linear mode (see section 3 below).

2) Determination of Number of Cysteines: Reduction and S-Pyridylethylation.

The number of cysteine residues was determined on the native peptide by reduction and S-pyridylethylation. 400 pmoles of native peptide were reduced in 40 μl of 0.5M Tris/HCl buffer, pH 7.5, containing 2 mM EDTA and 6 M guanidinium chloride in the presence of 2 μl of dithio-threitol (2.2M). The reaction medium was placed in a nitrogen atmosphere. After 60 min incubation in the dark, 2 μl of freshly distilled 4-vinylpyridine were added to the reaction which was incubated for 10 min at 45° C. in the dark and in a nitrogen atmosphere. The pyridylethylated peptide was then separated from the constituents of the reaction medium by reverse phase chromatography on a reverse phase Aquapore RP-300 C₈ analytical column (Brownlee™, 220×4.6 mm, 300 A) using a linear gradient of acetonitrile in the presence of 0.05% TFA from 2 to 52% for 70 minutes.

3) Mass Determination of the Native Peptide, S-Pyridylethylated Peptide and Proteolysed Fragments by MALDI-TOF Mass Spectrometry (Matrix Assisted Laser Desorption Ionisation—Time of Flight).

Mass measurements were made on MALDI-TOF Bruker Biflex mass spectrometry equipment (Bremen, Germany) in positive linear mode. The mass spectra were calibrated externally with a standard mixture of peptides of known m/z, respectively 2199.5 Da, 3046.4 Da and 4890.5 Da. The different products to be analysed were deposited on a thin layer of α-cyano-4-hydroxycinnamic acid crystals obtained by fast evaporation of a solution saturated in acetone. After drying in a slight vacuum the samples were washed in a drop of 0.1% trifluoroacetic acid before being placed in the mass spectrometer.

4) Sequencing by Edman Degradation

Automatic sequencing by Edman degradation of the native peptide, S-pyridylethylated peptide and various fragments obtained after the different proteolytic cleavage operations and detection of phenylthiohydantoin derivatives were performed on an AB1473A sequencer (PEApplied Biosystems Division of Perkin Elmer).

5) Proteolytic Cleavage

Confirmation of the peptide sequence in the C-terminal region: 200 pmoles of reduced, S-pyridylethylated peptide were incubated in the presence of 5 pmoles of endoproteinase-Lys-C (Acromobacter protease I, specific cleavage of the lysine residues on the C-terminal side (Takara, Otsu) following the conditions recommended by the supplier (10 mM Tris-HCl, pH 9 in the presence of 0.01% Tween 20. After stopping the reaction with 1% TFA the peptide fragments were separated by reverse phase HPLC on a column of Narrowbore DeltaPak™ HPIC₁₈ type (Waters Associates, 150×2 mm) in a linear gradient of acetonitrile from 2 to 60% in 80 min in 0.05% TFA at a rate of 0.2 ml/min and a constant temperature of 37° C. The fragments obtained were analysed by MALDI-TOF mass spectrometry and the peptide corresponding to the C-terminal fragment was sequenced by Edman degradation.

### EXAMPLE 3

Production of the Ard1 Peptide in S. cerevisiae Yeast.

1) Construction of the pEM2 Vector Permitting Expression and Secretion of the Ard1 Analog by the Yeast S. cerevisiae.

Using the heliomycin expression vector pSEA2 described by Lamberty et al. (1999, J. Biol. Chem., 274, 9320-9326), directed mutagenesis was performed by PCR to modify the codons Asp17 to Asn and Gly20 to Ala. A fragment carrying the MFA1 promoter, pre BGL2 and pro MFα1 sequences and the sequence encoding heliomycin as far as the SacII site was amplified by PCR with the oligonucleotides EM72 and EM89. The mutations of codons 17 and 20 were inserted the EM89 oligonucleotide.

| | |
|---|---|
| EM72 | 5′ GTAAATGCATGTATACTAAACTCACA 3′ |
| | SacII |
| EM89 | 5′ TTTTTTC\overline{C GCG G}CG CTT GCA CTC GGC GTT GCA GTT ACT |
| | (3′ CGC CGC GAA CGT GAG CCG CAA CGT CAA TGA |
| | Arg Arg Lys Cys GLu Ala Asn Cys Asn Ser |
| | AGT GTA GTT GAC GGC GC 3′ |
| | TCA CAT CAA CTG CCG CG 5′) |
| | Thr Tyr Asn Val Ala |

The PCR-amplified fragment was digested with the restriction enzymes SphI and SacII and cloned in the pSEA2 plasmid digested with the same enzymes and treated with alkaline phosphatase. The resulting pEM2 plasmid was controlled by restriction analysis and sequencing.

2) Transformation of a Yeast Strain S. cerevisiae by the pEM2 Plasmid.

The yeast strain TGY48.1 (MATα, ura3-Δ5n his, praI, prb1, prc1, cps1, Reichhart at al., 1992, Invert. reprod. Dev. 21, 15-24) was transformed using the PEM2 plasmid. The transformants were selected on a selective YNBG medium 0.5% supplemented with 0.5% casamino acids.

### EXAMPLE 4

Preparation of Heliomycin Analogues, pEM22, pEM24, PEM30, pEM31, pEM34, pEM35, pEM37, pEM46 and pEM48.

1) Construction of the pEM22 and pEM24 Vectors.

A synthetic fragment made up of the oligonucleotides EM25 and EM26 previously hybridised (heated to 100° C. and slow drop in temperature down to 25° C.) was cloned in the pSEA2 vector digested with BamHI and SalI (replacement of the 3′ end of the sequence coding for heliomycin, codon Ser34 as far as stop codon). This synthetic fragment BamHI-SalI contains the restriction sites XhoI and Nhe1. The resulting pEG01 vector was controlled by restriction analysis and sequencing.

| | |
|---|---|
| EM25: | 5′ GATCCACTCGAGTGCTAGCG 3′ |
| | XhoI NheI |
| EM26: | 5′ TCGACGCTAGCACTCGAGTG 3′ |
| | NheI XhoI |

A synthetic fragment BamHI-Sal1 made up of the previously hybridised oligonucleotides EM119 and EM120 was cloned in the pEG01 vector. The ligation reaction was digested with Xho1 to remove the plasmids which had not inserted into the synthetic EM119/EM120 fragment. The resulting pEM22 plasmid was controlled by restriction analysis and sequencing. An identical cloning strategy was used to construct pEM24 using the oligonucleotide pair EM127 and EM128.

| | |
|---|---|
| EM119 | 5′ GA TCC TTC ATT AAC GTT AAC TGT TGG TGT GAA ACC TGA TAG G 3′ |
| | Ser Phe Ile Asn Val Asn Cys Trp Cys Glu Thr |
| EM120 | 5′ TC GAC CTA TCA GGT TTC ACA CCA ACA GTT AAC GTT AAT GAA G 3′ |
| EM127 | 5′ GA TCC TTC TTG AAC ATT AAC TGT TGG TGT GAA ACC TGA TAG G 3′ |
| | Ser Phe Leu Asn Val Asn Cys Trp Cys Glu Thr |
| EM128 | 5′ TC GAC CTA TCA GGT TTC ACA CCA ACA GTT AAT GTT CAA GAA G 3′ |

2) Construction of the Vectors pEM30, pEM31, pEM34, pEM35, pEM46 and pEM48.

A synthetic fragment made up of the oligonucleotides EM25 and EM26 previously hybridised (heating to 100° C. and slow temperature drop down to 25° C.) was cloned in the pEM2 vector digested with BamHI and SalI (replacement of the 3′ end of the sequence encoding Ard1, Ser34 codon as far as stop codon). This synthetic fragment BamHI-Sal1 contains the restriction sites XhoI and Nhe1. The resulting pEM16 vector was controlled by restriction analysis and sequencing.

A synthetic fragment BamHI-SalI made up of the previously hybridised oligonucleotides EM135 and EM136 was cloned in the pEM16 vector. The ligation reaction was digested with Xho1 to remove the plasmids which did not insert into the synthetic fragment EM135/EM136. The resulting pEM30 plasmid was controlled by restriction analysis and sequencing. An identical cloning strategy was used for the constructions of pEM31 (EM117/EM118), pEM34 (EM127/EM128), pEM35 (EM129/EM130), pEM46 (EM158/EM159), pEM48 (EM162/EMI63).

| | |
|---|---|
| EM117 | 5′ GA TCC TTC GCT AAC GTT AAC TGT TGG TGT CAA ACC TGA TAG G 3′ |
| | Ser Phe Ala Asn Val Asn Cys Trp Cys Gln Thr . . |
| EM118 | 5′ TC GAC CTA TCA GGT TTG ACA CCA ACA GTT AAC GTT AGC GAA G 3′ |
| EM129 | 5′ GA TCC TTC TTG AAC GTT AAC TGT TGG TGT GAA ACC TGA TAG G 3′ |
| | Ser Phe Leu Asn Val Asn Cys Trp Cys Glu Thr . . |
| EM130 | 5′ TC GAC CTA TCA GGT TTC ACA CCA ACA GTT AAC GTT CAA GAA G 3′ |
| EM135 | 5′ GA TCC TTC GCT AAC GTT AAC TGT TGG TGT GAA AGA TGA TAG G 3′ |
| | Ser Phe Ala Asn Val Asn Cys Trp Cys Glu Arg . . |
| EM136 | 5′ TC GAC CTA TCA TCT TTC ACA CCA ACA GTT AAC GTT AGC GAA G 3′ |
| EM158 | 5′ GA TCC TTC TTG AAC CTT AAC TGT TGG TGT CAA ACC TGA TAG G 3′ |
| | Ser Phe Leu Asn Val Asn Cys Trp Cys Gln Thr . . |
| EM159 | 5′ TC GAC CTA TCA GGT TTG ACA CCA ACA GTT AAC GTT CAA GAA G 3′ |
| EM162 | 5′ GA TCC TTC TTG AAC GTT AAC TGT TGG TGT GAA AGA TGA TAG G 3′ |
| | Ser Phe Leu Asn Val Asn Cys Trp Cys Glu Arg . . |
| EM163 | 5′ TC GAC CTA TCA TCT TTC ACA CCA ACA GTT AAC GTT CAA GAA G 3′ |

3) Construction of the Expression Vector pEM37.

From the expression vector of heliomycin pSEA2, directed mutagenesis was performed using PCR to modify the Asp1 codon to Asn. A fragment carrying the end of the pro sequence of MFα1 and the sequence encoding heliomycin was amplified by PCR with the oligonucleotides EM137 and EM53. The mutation of the Asp1 codon to Asn was inserted in the olignucleotide EM137.

| | |
|---|---|
| EM53 | 5′ CCTGGCAATTCCTTACCTTCCA 3′ |
| | HindIII |
| EM137 | 5′ TTTTTTA AGC TTG GAT AAA AGA AAC AAG TTG ATT GGC AG 3′ |
| | Ser Leu Asp Lys Arg Asn Lys Leu Ile Gly |

The PCR-amplified fragment was digested with the restriction enzymes HindIII and SalI and simultaneously cloned with a SphI-HindIII fragment of 1.2 kb carrying the MFα1 promoter, the pre sequence of BGL2 and pro sequence of MFα1 as far as the HindIII site in the pTG4812 vector (Michaud et al., 1996, FEBS Lett., 395, 6-10) digested with SphI and SalI and treated with alkaline phosphate. The resulting pEM37 plasmid was controlled by restriction analysis and sequencing.

### EXAMPLE 5

Screening Tests made on Heliomycin Analogues.

1) Cultures

The yeast clones transformed by the expression plasmids of heliomicin and its analogues were cultured in selective medium (50 ml YNBG+0.5% casamino acid) for 72 hours under stirring at 29° C. After centrifuging at 4000 g for 30 min at 4° C. the supernatants were acidified to pH3 with acetic acid.

The supernatants were then deposited on a reverse phase 360 mg carrier Sep-Pak™ (Waters Associates) equilibrated with acidified water (0.05% TFA). The hydrophilic molecules were removed by simply washing with acidified water. The peptides were eluted with a 60% acetonitrile solution prepared in the 0.05% TFA. The fraction eluted at 60% acetonitrile was vacuum dried to remove the acetonitrile and TFA and then reconstituted in 1 ml 0.05% TFA water before undergoing purification.

2) Purification by High Pressure Liquid Chromatography (HPLC) on Reverse Phase Column.

Depending upon the production level obtained for each analogue, the equivalent of 5 to 20 ml of pre-purified supernatant was analysed by reverse phase chromatography on an Aquapore RP-300 C₈ semi-preparation column (Brownlee™, 220×7 mm, 300 A), elution was performed on an acetonitrile gradient in 0.05% TFA from 2% to 22% in 5 minutes, then 22 to 40% in 30 minutes after a 2-minute isocratic at 22%, at a constant rate of 1.4 ml/min. The fractions eluted between 27% and 38% acetonitrile were collected manually following absorbency variation at 225 nm.

3) Control of Analogue Mass

1 μl of majority fractions was diluted 2 times in water acidified with 0.05% TFA and analysed by MALDI-TOF mass spectrometry. The fraction whose measured mass corresponds to theoretical mass was vacuum dried and reconstituted by adding one volume of ultrapure water calculated as described in the following paragraph.

4) Quantification of the Analogue for Activity Tests

A calibration curve of the semi-preparation Aquapore RP-300 C₈ column was made by injecting 5, 10, 20 and 25 mg heliomycin. Integration, calculation of areas and slant were made using Millenium software (Waters). Subsequently, the quantification of the analogues (in μg) was calculated by automatic integration of the chromatogram peak corresponding to the analogue, using this software. The take-up volume of the sample after evaporation was calculated in relation to the quantification so obtained so as to adjust the peptide concentration to 1 μg/μl.

5) Anti-Candida albicans and Anti-Aspergillus fumigatus Activity Tests.

The anti-Candida albicans and anti-Aspergillus fumigatus activities of the different analogues were assessed using a growth inhibition test in liquid medium made in 96-well microplates. The activity of the purified peptides was tested for different dilutions of each peptide and was compared with those of heliomycin and Ard1 quantified under the same conditions.

—Anti-Candida albicans Test

The activity test was made directly on yeasts derived from a stock frozen at −80° C., in Sabouraud medium containing 15% glycerol. The density of the yeasts in the stock was adjusted to an optical density of 0.4 OD at 600 nm. After slow thawing at room temperature, the yeast suspension was reduced by dilution to an optic density of 1 mOD at 600 nm in Sabouraud medium, and 90 μl of this dilution were deposited in the wells of microtitration plates in the presence of 10 μl of sample to be tested. Control samples were systematically made in which 10 μl of sample were replaced by 10 μl of sterile water. Media sterility was controlled by incubating 10 μl sterile water in the presence of 90 μl of medium. The samples were incubated at 30° C. for 40 h under slight stirring and the antifungal activity was quantified by measuring optic density at 600 nm.

—Anti-Aspergillus fumigatus Test

The spores of Aspergillus fumigatus were derived from a stock frozen at −80° C., containing 10⁷ spores/ml in a 25% glycerol solution. After slow thawing at room temperature, the spores were placed in suspension in PDB culture medium (12 g Potato Dextrose Broth medium, per 1 demineralised water). 10 μl of each sample were deposited in the wells of microtitration plates in the presence of 90 μl PDB culture medium supplemented with tetracycline (100 μg/ml) and cefotaxime (1 μg/ml) containing the spores (at a final concentration of 1000 spores/well). Control cultures were systematically made in which 10 μl of sample were replaced by 10 μl of sterile water. Media sterility was controlled by incubating 10 μl sterile water in the presence of 90 μl of medium. The samples were incubated at 37° C. for 24 h to 48 h in a humid atmosphere, and the antifungal activity was quantified by a score of 0 to 9 taking germination account; the size and morphology of the hyphs were determined under the binocular microscope. The minimal inhibiting concentration (MIC) was 4.

6) Control of Quantification

The solutions of peptides used for the activity tests were systematically subjected to quantification control by injecting 10 μl under HPLC into a Narrowbore Delta-Pack™ HPI C₁₈ column previously calibrated with 2, 5, 7.5 and 10 μg heliomycin. The quantity of peptides effectively deposited in the wells was readjusted whenever necessary for interpretation of results.

### EXAMPLE 6

In Vivo Efficacy

1) Method—Candida albicans Infected Model

Heliomycin and its analogues were tested for in vivo antifungal activity in a model infected with Candida albicans, lethal in mice. The pathogenic agent Candida albicans (IHEM 8060 strain) was inoculated by intravenous route (i.v.) at a dose of 2.5×10⁶ CFU/mouse. the peptides were administered by i.v. route in 4 injections 6 h, 24 h, 48 h and 72 h after infection. Assessment criteria for activity were evaluation of survival and morbidity at 7 days. The morbidity scores, which take into account general state of health (condition of fur, mobility, hydration) were determined for each mouse with values ranging from 0 to 5 and defined below: 0=dead, 1=moribund, 2=very ill, 3=ill, 4=slightly ill, 5=healthy. The sum of the individual scores was calculated for each group, a score of 50 for a group of 10 mice meaning that all the mice were healthy.

2) Comparison of the Activities of Ard1 and Heliomycin in the Candidiasis Infected Model.

Following a standard protocol, groups of 10 Swiss OF1 male mice weighing 12 g were infected via i.v. route with a dose of 2.5×10⁶ CFU/mouse. Heliomycin and Ard1 were administered by i.v. route in 4 injections, 6 h, 24 h, 48 h and 72 h after infection. For each peptide, 2 doses were tested: 10 mg/kg and 30 mg/kg. A placebo group was injected with peptide solvent, 0.9% sodium chloride.

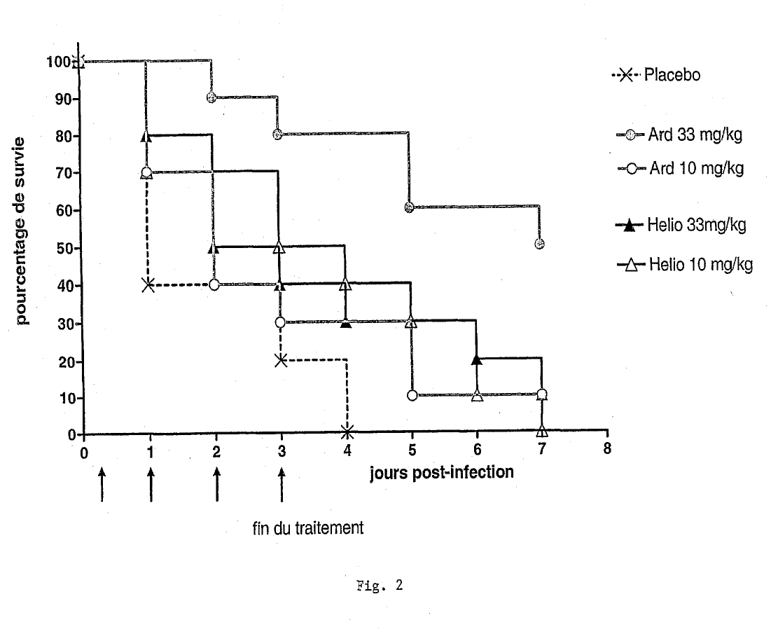

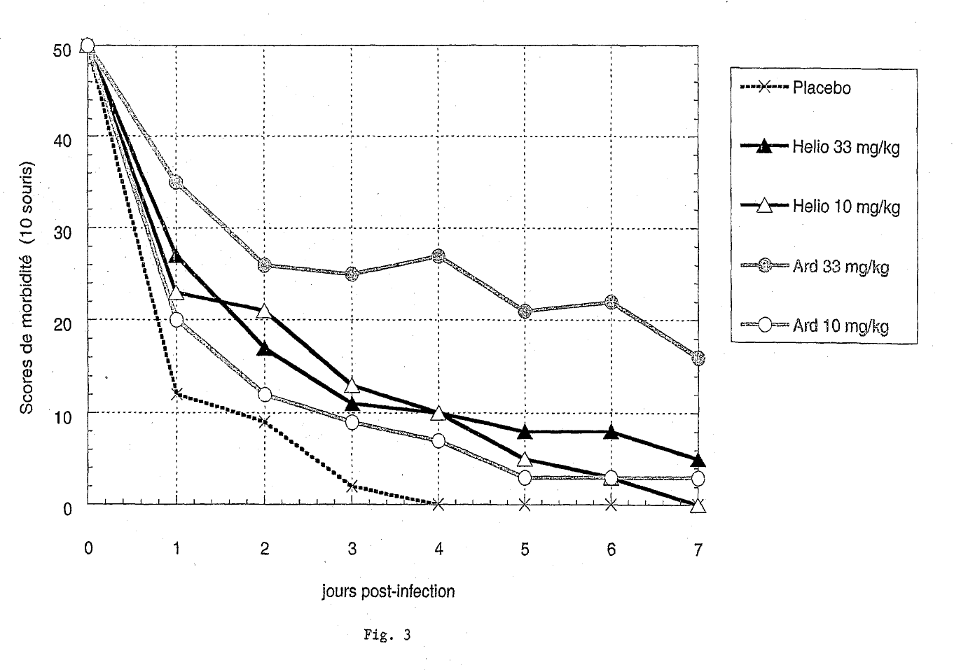

FIGS. 2 and 3 respectively show the survival rate and morbidity scores (10 mice) in relation to the number of post-infection days.

In this very severe infection model, 100% lethal at post-infection day 4, 60% of mice in the placebo group were dead on the first day after infection.

It was observed that Heliomycin administered at 10 and 30 mg/kg has no significant effect on survival rate, even though the curves are above the relative curve of the placebo group. Median mortality occurred in the groups treated with doses of 10 and 30 mg/kg Heliomycin respectively, at 48 h and 60 h after infection, and the morbidity scores were 0/50 and 5/50 at day 7.

Under these conditions, the Ard1 peptide administered at the dose of 30 mg/kg delayed the onset of the first death by 24 h. 5 mice out of 10 were still alive on day 7 with a morbidity score of 16/50. Comparison of the survival curves (Meier-Kaplan) using the logrank statistical test led to finding a significant difference between the placebo group and the group treated with Ard1 at 30mg/kg (p<0.001).

Ard1 administered at the dose of 10 mg/kg did not make it possible to improve survival and general condition of the mice, 50% of mice being dead 2 days after infection.

3) Comparison of the Activities of Ard1 and the Analogues pEM24, pEM30, pEM31 and pEM35 in the Candidiasis Infected Model.

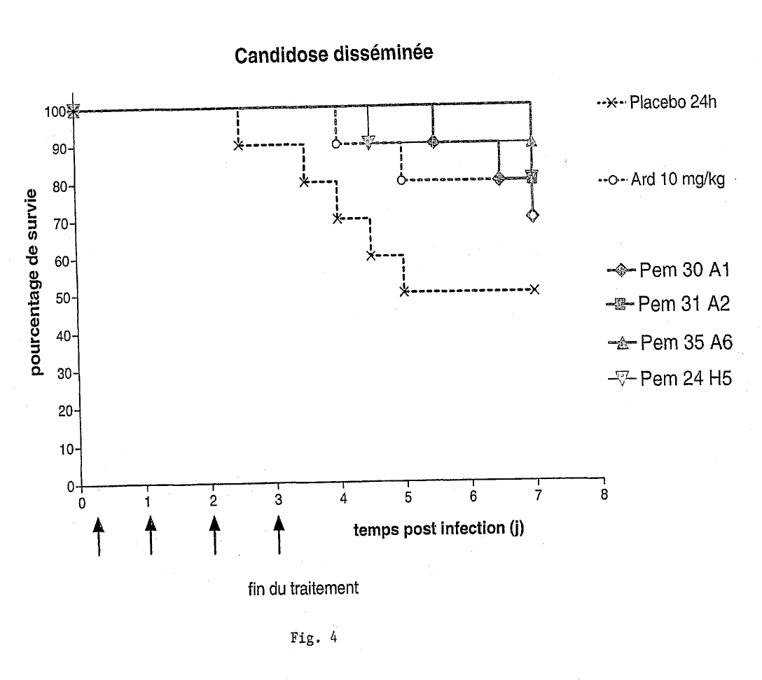

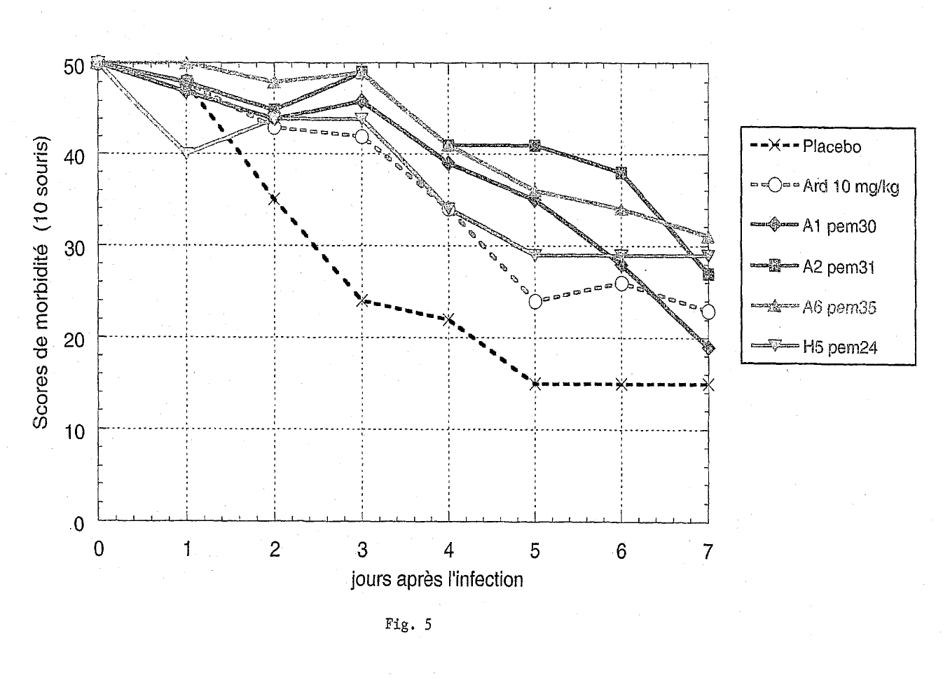

FIGS. 4 and 5 respectively show the survival rate and morbidity scores (10 mice) in relation to post-infection days.

In this experiment, inoculation of the mice with 2.5.10⁶ CFU/mouse was 50% lethal at day 5 in the group which received placebo treatment. The first deaths occurred on post-infection day 2.5 and median mortality occurred on post-infection day 5. On day 7, 5 mice were alive and the morbidity score 15/50.

Ard1, at a dose of 10 mg/kg, delays the onset of the first death by 1.5 days. 8 mice were still alive on post-inoculation day 7. The survival curve was not statistically different, however, from that of the placebo group (p=0.2516).

The four peptides tested at the dose of 10 mg/kg, pEM24 (H5), pEM30 (A1), pEM31 (A2) and pEM35 (A6), are more active than Ard1: the time of onset of the 1^{st} death and the number of mice alive on day 7 were respectively 3 days and 7 mice for the group treated with pEM24 (H5), 4 days and 7 mice for the group treated with pEM30 (A1), 5.5 days and 8 mice for the group treated with pEM31 (A2) and 7 days and 9 mice for the group treated with pEM35 (A6). With each of these peptides it was possible to maintain the mice in a good general state of health for the 3 first days, the morbidity scores lying between 42 and 48/50 on day 3, compared with 22/50 for the group which received the placebo. The condition of the mice declined 24 h after the 4^{th} injection. Only the group treated with pEM31 maintained a morbidity score that was higher than 40/50 for 5 days.

The statistical comparison of the survival curves with the curves for the placebo group shows a significant difference for the group treated with pEM35 with p being 0.041.

Statistical comparison of the survival curves with those of the placebo group shows a significant difference for the group treated with pEM31 on day 8 with p being 0.0195.

The relative activities of the peptides are the following: pEM31≧pEM35≧pEM30≧pEM24≧Ard1.

4) Comparison of the Activities of Ard1 and the Analogues pEM31, pEM35, pEM37, pEM46 and pEM51 Administered in 5 mg/ml in the Candidiasis Infected Model.

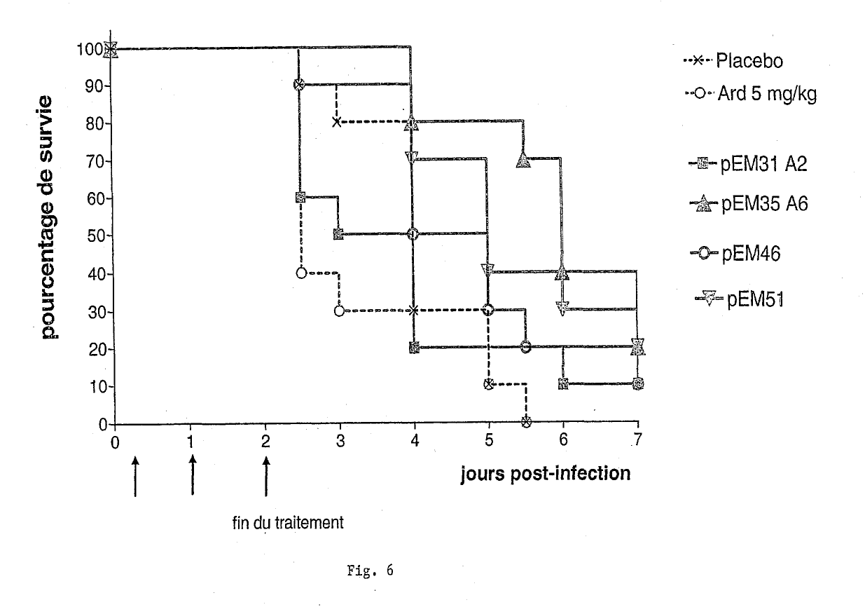

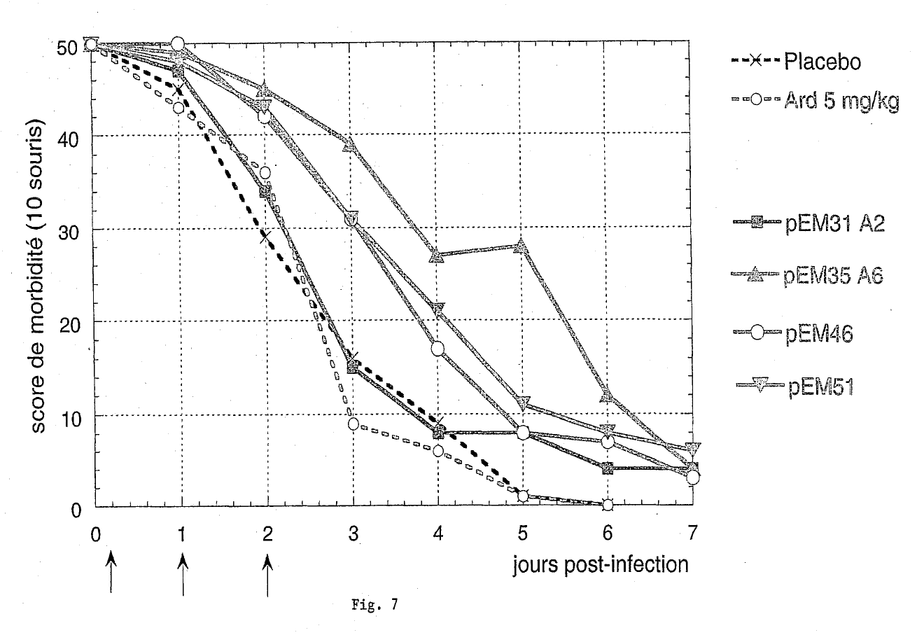

FIGS. 6 and 7 respectively show the survival rate and morbidity scores (10 mice) in relation to post-infection days.

In this experiment, inoculation of Swiss OF1 mice weighing 15 g with 3.10⁶ CFU of Candida albicans induces 50% mortality on day 4 after infection in the group treated with the placebo. The first deaths occur 2.5 days after infection, and 100% of the mice were dead on day 5.5.

Treatment with Ard1 and with pEM31 and pEM46, administered in three i.v. injections at the dose of 5 mg/kg does not significantly increase mouse survival relative to placebo treatment. However, treatment with pEM45 delays deterioration in state of health of the mice with a morbidity score of 31/50 3 days after infection, compared with 9/50 for mice in the placebo group.

At this dose, treatments with pEM51 and pEM35 delayed the onset of the 1^{st} death by 1.5 days; median mortality occurred on post-infection days 5 and 6 respectively for the groups treated with pEM51 and pEM35. Statistically, analysis of the survival curves at day 7 shows a significant difference relative to the placebo group with p being 0.015 for the group treated with pEM51 and p being 0.0004 for the group treated with pEM35. The general state of health of the mice treated with pEM35 is better than that of the mice who were given the other treatments, with a morbidity score remaining at 28/50 up to post-infection day 5 compared with a score of 11/50 for the mice who were given pEM51 and a score of 1/50 for the mice who received a placebo.

Overall, in this candidiasis infected model, the antifungal activity of pEM35 was greater than that of pEM51 which itself was greater than the activity of pEM46. At the dose used of 5 mg/kg, the Ard1 and pEM31 molecules are not effective.

5) Activity of the pEM35 Analogue in the Candidiasis Infected Model.

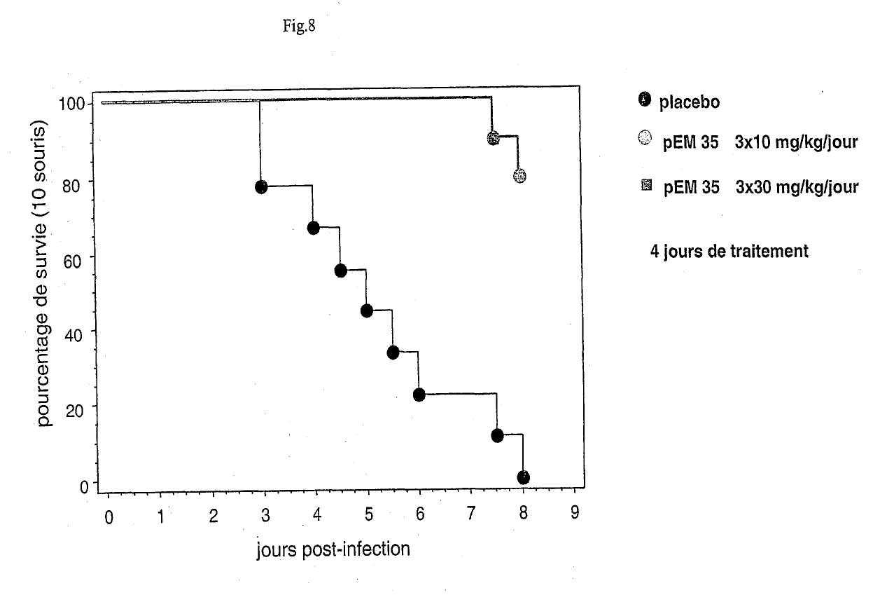

FIG. 8 shows the survival rate (10 mice) in relation to post-infection days. In this experiment, inoculation of the mice with 2.5.10⁶ CFU/mouse was 50% lethal at day 5 and 100% at day 8 for mice in the placebo group. The first death occurred 3 days after infection. The morbidity score fell rapidly below 30/50 (25/50 at day 2.5).

The pEM35 peptide was administered at doses of 10 and 30 mg/kg/injection with 3 daily doses for 4 days (1 h, 5 h and 10 h post-infection on day 0; at 8 h, 14 h and 20 h on days 1, 2 and 3), i.e., daily doses totalling 30 and 90 mg/kg.

With this administration schedule, pEM35 was able to delay the onset of the first death by 4 and a half days for both doses. On post-infection day 8, a respective survival rate of 80% and 90% was observed for the mice treated with doses of 30 and 90 mg/kg/day. The mice remained in good state of health until day 7, with a morbidity score which remained above 40/50. On day 8, the scores fell to 30/50. No major difference was seen between the groups treated with pEM35 at the low dose of 30 mg/kg/day and the strong dose of 90 mg/kg/day.

The survival curves in relation to the groups treated with pEM35 are statistically different from the curve for the placebo group (p<0.001 for both doses).

6) Method—Scedosporium inflatum Infected Model.

Swiss OF1 mice weighing 22 g were infected by intravenous route (i.v.) with a lethal dose of Scedosporium inflatum (FSSP 7908 strain cultured on Malt Agar gelose for 7 days at 37° C.). The infecting dose was 7.10⁶ spores per mouse, injected in a volume of 100 μl via the lateral tail vein.

Peptides pEM35 and pEM51 were administered continuously using ALZET 1003D osmotic pumps (flow rate: 0.97 μl/h; volume: 93 μl; infusion time: 4 days) and 1007D pumps (flow rate: 0.47 μl/h; volume: 100 μl; infusion time: 8 and half days) with intraperitoneal insertion

Groups of 8 infected mice were treated either with:

a) a placebo: 0.9% NaCl via 1007D pumps with intra-peritoneal insertion (i.p.);

b) not treated;

c) with pEM51 delivered i.p. by 1007D pumps at a dose of 30 mg/kg for 8 days, corresponding to a theoretical equilibrium plasma concentration of 0.3 μg/ml;

d) with pEM51 delivered i.p. by 1003D pumps at a dose of 60 mg/kg for 4 days, corresponding to a theoretical equilibrium plasma concentration of 0.6 μg/ml;

e) with pEM35 delivered i.p. by 1003D pumps at a dose of 35 mg/kg for 4 days, corresponding to a theoretical equilibrium plasma concentration of 0.35 μg/ml.

7) Activity of the Analogues pEM35 and pEM51 Delivered under Continuous Infusion in a Scedosporiosis Infected Model

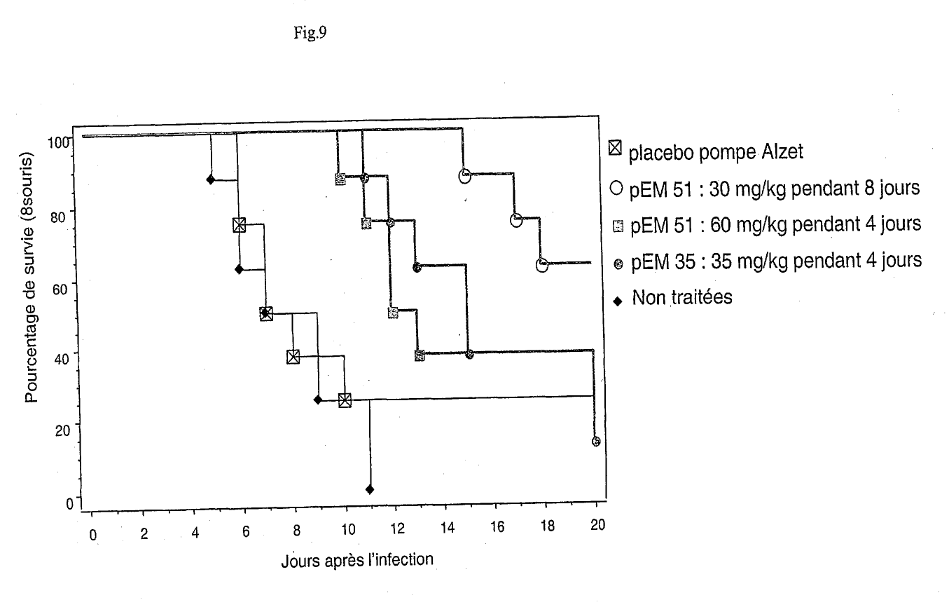

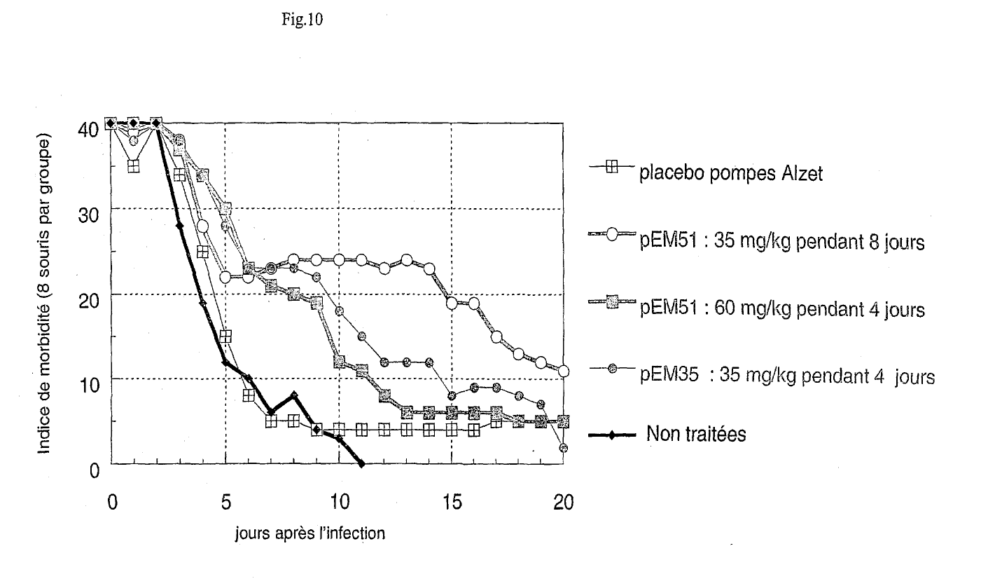

FIGS. 9 and 10 respectively show the survival rate and morbidity score (8 mice) in relation to post-infection days.

In this model of invasive scedosporiosis, inoculation of a dose of 7.10⁶ spores of Scedosporium inflatum was 50% lethal on post-infection day 7. The first death occurred at 5 days and 6 days respectively after infection for the control mice group (infected, non-treated) and the placebo group (infected and with Alzet pumps). 100% mortality was observed on day 11 in the control group and 75% mortality on day 20 for the placebo group. The state of health of the mice deteriorated rapidly on and after post-infection day 3 with a morbidity score for these two groups of 28/40 and 34/40 on day 3 and 6/40 and 7/40 on post-infection day 7. Signs of encephalitis occurred on post-infection day 4.

Treatment with pEM51 at a dose of 30 mg/kg for 8 days made it possible to delay the onset of the first death by 9 days. On day 20, 5 mice out of 8 were still alive. The morbidity score decreased on and after post-infection day 4 (28/40) corresponding to the onset of signs of encephalitis, and stabilized at 24/40 on post-infection day 5 until post-infection day 14. The state of health of the mice deteriorated gradually thereafter with a score of 11/40 on post-infection day 20. The mortality curve is statistically different from those for the control and placebo groups (logrank: p=0.0027).

Under treatment with pEM51 at a dose of 60 mg/kg for 4 days, the onset of the first death was delayed by 4 days. 50% mortality was observed on post-infection day 12, that is a 5-day delay in relation to the contra and placebo groups. On day 20, 3 mice out of 8 were still alive. The morbidity score decreased as from post-infection day 5 (30/40), corresponding to the onset of signs of encephalitis, and gradually fell to a score of 6/40 on post-infection day 14. The mortality curve is statistically different from those for the control and placebo groups (logrank: p=0.0176).

Under treatment with pEM35 at a dose of 35 mg/kg for 4 days it was possible to delay the onset of the first death by 5 days. 50% mortality was observed on post-infection day 15, that is an 8-day delay relative to the control and placebo groups. On day 20, 1 mouse out of 8 was still alive. The morbidity score decreased on an after post-infection day 5 (28/40) corresponding to the onset of signs of encephalitis, and gradually fell to a score of 8/40 on post-infection day 15. The mortality curve is statistically different from those for the control and placebo groups ( logrank: p=0.0177).

In this model, pEM51 administered at a dose of 30 mg/kg for 8 days showed very good therapeutic efficacy in terms of survival. The administration of a dose twice as high (60 mg/kg) over a period twice as short distinctly reduced the efficacy of pEM51. However, during the first 4 treatment days, the morbidity score of the group treated with the dose of 30 mg/kg was substantially lower than in the group treated with the dose of 60 mg/kg. The administration of a dose of 60 mg/kg for 8 days should therefore further improve the therapeutic efficacy of pEM51.

pEM35 at the dose of 35 mg/kg for 4 days showed the same efficacy as pEM51 at the dose of 60 mg/kg for 4 days. The therapeutic activity of pEM35 in this Scedosporiosis model is therefore at least equivalent to that of pEM51.

8) Acute Toxicity Study of pEM35 and pEM51 in Mice

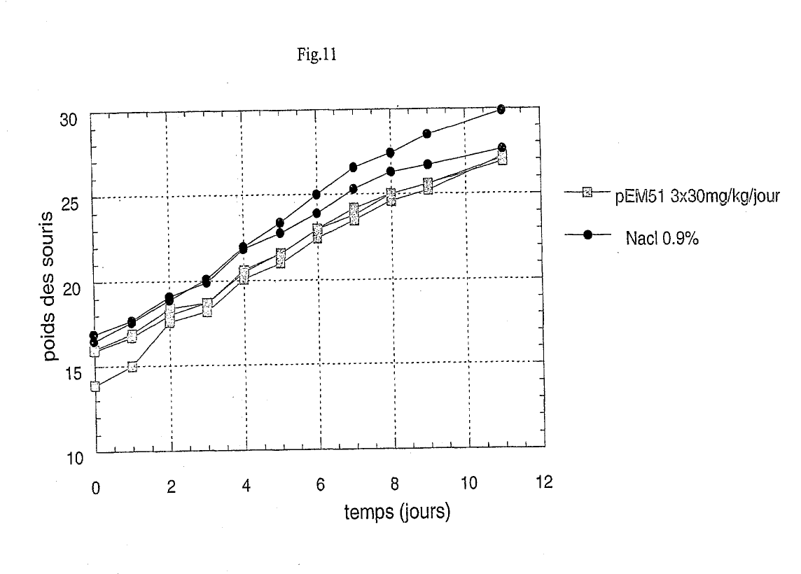

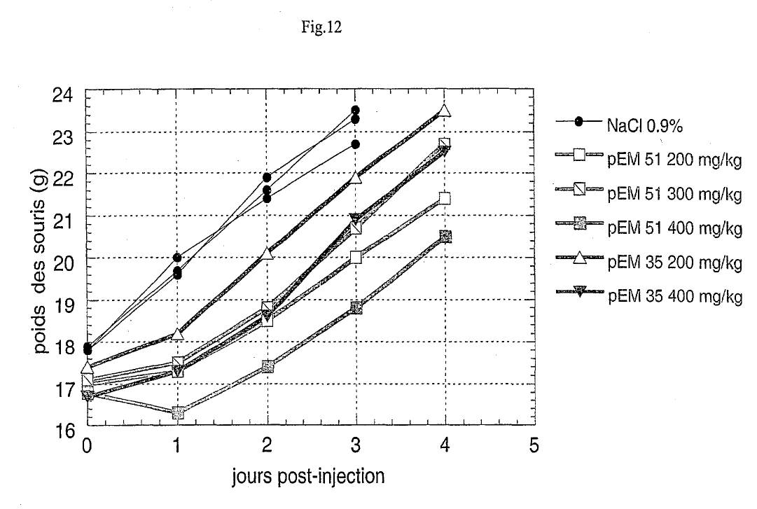

FIGS. 11 and 12 show the weight changes in treated healthy mice in relation to time.

During therapeutic efficacy tests in mice, no acute toxicity was observed with intravenous administration of pEM35 and pEM51 dissolved in 0.9% NaCl, in injections of 30 mg/kg repeated at 30-minute intervals given 3 times daily for 3 days.

The weight changes in healthy mice treated with 3 daily doses of 30 mg/kg of pEM51 for 3 days were similar to those for mice injected with 0.9% NaCl.

The acute toxicity of pEM35 and pEM51 in a single dose by intravenous route was tested in Swiss OF1 male mice weighing 17-18 g in doses of 200, 300 and 400 mg/kg. The peptides were solubilised in a 0.9% NaCl solution; the injected volume was 150 μl injected in 45 seconds via the lateral tail vein.

All mice showed vasodilatation associated with prostration. The state of heath of the mice returned to normal 20 to 40 minutes after injection depending upon the dose.

The weight change curves over 4 days show slight delayed growth on the day after the injection, of approximately 1 g for the mice given pEM35 at doses of 200 and 400 mg/kg or pEM51 at doses of 200 and 300 mg/kg; and of approximately 2 g for the mouse given pEM51 at the dose of 400 mg/kg. The weight curve then returned to normal for all mice.

### EXAMPLE 7

Spectrum of the Antifungal Activity of Ard1 and the Analogues pEM31, pEM35, pEM46, pEM48 and pEM51.

1) Test to Detect Activity against Filamentous Fungi.

The antifungal activity was detected by a growth inhibition test in a liquid medium.

The filamentous fungi (A. fumigatus, A. flavus and A. terreus, donated by Dr. H. Koenig, Hôpital Civil, Strasbourg; and S. prolificans and F. solani donated by Drs. J. Meis and J. Mouton, University hospital, Microbioolgy Department, Nijmegen, Netherlands) were seeded on Malt-Agar slant gelose (Biomerieux) and incubated 7 days at 37° C.

The spores were then collected with 10 ml YPG medium containing 0.05% Tween 20 and filtered through a gauze. The spores were centrifuged 10 min at 1700 rpm, the residue was collected in YPG (1 g Yeast extract, 1 g Peptone, 3 g Glucose per 1 l).

The suspension was counted with a Coverslide and adjusted to 10⁴ spores/ml.

100 μl of peptide dilutions (concentration of 50 with 0.097 μg/ml peptide) were deposited in microtitration plates. 100 μl with 10⁴ spores/ml of filamentous fungi, i.e. 1000 spores, were then added.

The test plates were incubated 48 h at 37° C.

Determination of minimum inhibiting concentrations (MIC) was made by observing well cover rate. The MIC score was set at 50% well covering.

2) Test to Detect Anti-Yeast Activity

Candida yeasts (C. albicans, C. glabrata, C. dubliensis, C. tropicalis, C. kefyr, C. krusei and C. parapsilosis—donated by Dr. H. Koenig, Hôpital Civil, Strasbourg), fluconazole-resistant C. albicans (n^{o}245962, n^{o}2332, n^{o}246335 and n^{o}3552, donated by Drs. J.Meis and J. Mouton, University Hospital, Microbiology Department, Nijmegen, Netherlands), and Cryptoccocus neoformans (donated by Dr. H. Koenig, Hôpital Civil, Strasbourg) were seeded on Sabouraud-Cloramphenicol Agar slant gelose (Biomerieux) and left to incubate for 24 h at 30° C. (Candida sp.) and for 72 h at 37° C. (Cryptoccocus neoformans).

Some yeast colonies were placed in suspension in liquid Sabouraud medium (Biomerieux) to obtain a final concentration of 0.1 OD at 600 nm corresponding to 2.5.10⁶ yeasts/ml.

The yeast suspension was adjusted to 5.10³ yeast/ml in Sabouraud medium.

100 μl of peptide dilutions (concentration of 50 with 0.097 μg/ml peptide) were deposited in microtitration plates. After adding 100 ml of yeast suspension with 5.10³ yeast/ml i.e. 500 yeasts, the test plates were incubated 24 h at 30° C. (Candida) under slow shaking or 72 h at 37° C. (Cryptoccocus).

Determination of minimal inhibiting concentrations (MIC) was made by measuring absorbency at 600 nm using a spectrophotometer-microtitration plate reader. The MIC score was set at a growth inhibition rate of 50%.

3) Test to Detect Activity against Phytopathogens: Alternaria brassicola and Neurospora crassa.

100 μl of peptide dilutions (concentration 50 with 0.097 mg/ml peptide) were deposited in microtitration plates.

After adding 100 μl of frozen spores with 10⁴ spores/ml of A. brassicola and N. crassa (donated by Dr. Bullet, IBMC, Strasbourg) the test plates were incubated 48 h at 30° C.

Determination of minimal inhibiting concentrations (MIC) was made by observing well cover rate. The MIC was set at 50% well covering.

Table 3 below shows the MIC scores for Ard1 and its analogues (μg/ml) against yeasts and filamentous fungi. Tables 4 and 5 below show the respective MIC scores of the analogues pEM35 and pEM51 (μg/ml) against fluconazole-resistant strains of Candida albicans yeast and against filamentous fungi.

TABLE 3

| Yeasts | Ard-1 | pEM31 | pEM48 | pEM51 | pEM46 | pEM35 |
|---|---|---|---|---|---|---|
| C. albicans | 3.125- | 3.125- | 1.56 | 1.56- | 1.56- | 1.56 |
| | 6.5 | 6.25 | | 3.125 | 3.125 | |
| C. tropicalis | 6.25- | 6.25 | 3.125 | 3.125 | 3.125 | 1.56 |
| | 12.5 | | | | | |
| C. glabrata | >25 | >25 | >25 | >25 | >25 | >25 |
| C. parapsilosis | 3.125- | 1.56 | 0.78 | 0.78- | 0.78 | 0.78- |
| | 6.25 | | | 1.56 | 1.56 | 1.56 |
| C. dubliensis | 1.56- | 6.25 | 1.56- | 1.56- | 3.125 | 0.78- |
| | 3.125 | | 3.125 | 3.125 | | 1.56 |
| C. kefyr | >25 | >25 | 25 | >25 | >25 | >25 |
| C. krusei | 3.125 | 3.125 | 1.56 | 1.56 | 1.56 | 1.56 |
| C. neoformans | 12.5- | 12.5 | 3.125- | 1.56 | 6.25 | 6.25 |
| | 25 | | 6.25 | | | |
| Filam. fungi | | | | | | |
| A. fumigatus | 12.5 | 6.25- | 6.25- | 6.25- | 3.125 | 6.25 |
| | | 12.5 | 12.5 | 12.5 | | |
| A. flavus | 6.25- | >25 | 6.25- | 6.25 | 12.5- | 3.125 |
| | 12.5 | | 12.5 | | 25 | |
| A. terreus | 1.56- | 3.125- | 3.125- | 3.125 | 6.25 | 6.25 |
| | 3.125 | 6.25 | 6.25 | | | |
| A. brassicola | >25 | >25 | 12.5 | >25 | 25 | >25 |
| N. crassa | 0.097 | <0.048 | 0.39 | 0.195 | 0.195 | <0.048 |

TABLE 4

| C. albicans | pEM51 | pEM35 | ampho B | fluconazole | itraconazole |
|---|---|---|---|---|---|
| n° 245962 | 0.78- | 3.125- | 0.125 | > at 64 | 1-0.5 |
| | 0.39 | 1.56 | | | |
| n° 2332 | 1.56 | 1.56- | 0.25 | > at 64 | 0.5-0.25 |
| | | 0.79 | | | |
| n° 246335 | 1.56- | 3.125- | 0.0625 | > at 64 | 0.5-0.25 |
| | 0.78 | 1.56 | | | |
| n° 3552 | 1.56- | 3.125- | 0.125- | > at 64 | 1-0.5 |
| | 0.78 | 1.56 | 0.0625 | | |

TABLE 5

| Filament. fungi | Peptide | MIC (mg/ml) |
|---|---|---|
| FASF 5161 | pEM35 | 6.25-3.125 |
| A. fumigatus | pEM51 | 3.125-1.56 |
| | ampho B | 0.5 |
| FASF V02-31 | pEM35 | 12.5-6.25 |
| A. fumigatus | pEM51 | 12.5-6.25 |
| | ampho B | 1-0.5 |
| FSSP 7902 | pEM35 | 0.39-0.19 |
| S. prolificans | pEM51 | 0.19-0.09 |
| | amphoB | >16 |
| FSSP 7908 | pEM35 | 0.19-0.09 |
| S. prolificans | pEM51 | 0.09-0.048 |
| | ampho B | >16 |
| FFUS 8591 | pEM35 | 3.125-1.56 |
| F. solani | pEM51 | 0.78-0.39 |
| | ampho B | >16 |

### EXAMPLE 8

Fungicidal Kinetics of the Peptides pEM35 and pEM51 against Candida albicans IHEM 8060.

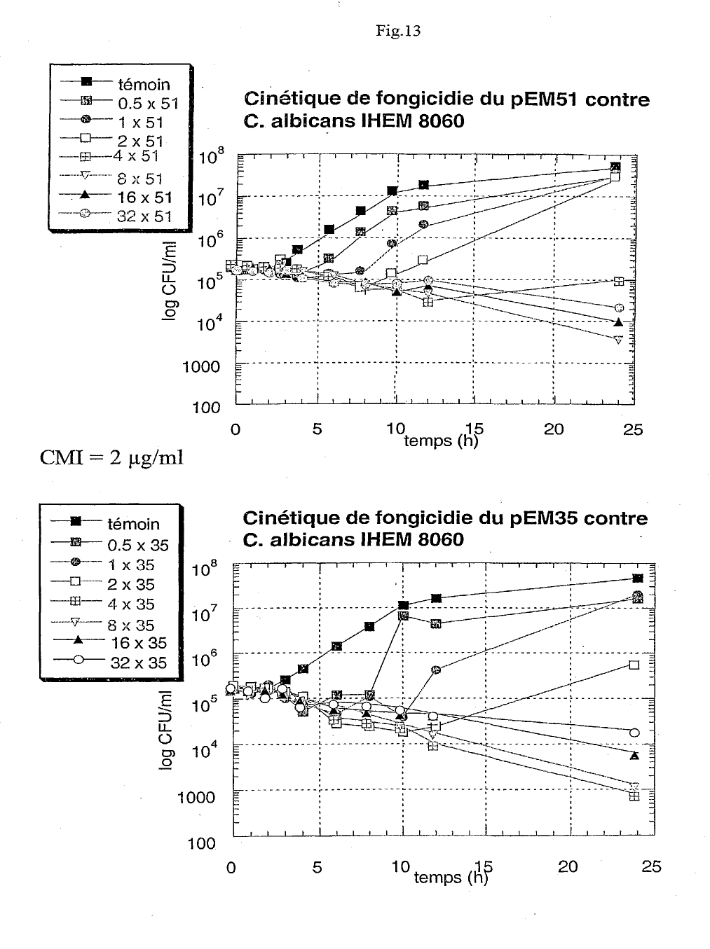

FIG. 13 shows the fungicidal kinetics of the pEM35 and pEM51 peptides against Candida albicans IHEM 8060.

The test was conducted in accordance with the protocol descried by Klepser et al. (Antimicrob Agents Chemother, May 1998, 42(5) :1207-12 “Influence of test conditions on antifungal time-kill curve results: proposal for standardized methods”). The strains of Candida albicans yeasts used were the same as those previously used for the test to detect anti-yeast activity (yeasts donated by Dr. Koenig, Hôpital Civil, Strasbourg).

The yeast strains were seeded on Sabouraud-Chloramphenicol gelose and left to incubate for 24 h to 48 h at 30° C. Some yeast colonies were placed in suspension in 4 ml of liquid Sabouraud medium (Biomerieux) and then incubated under overnight stirring at 30° C.

The yeast suspension was adjusted to 1.10⁶-5.10⁶ yeasts/ml in fresh Sabouraud. A dilution of 1:10 was prepared by adding 1 ml of the yeast suspension to 9 ml of Sabouraud-Chloramphenicol (Biomerieux) containing or not containing (control) a defined quantity of pEM35 or pEM51 peptide. The yeast concentration in the initial inoculum was therefore 1.10⁵-5.10⁵ yeasts/ml.

The pEM35 and pEM51 peptides were tested on a concentration range extending from 1 μg/ml to 64 μg/ml. Each of the solutions was incubated at 35° C. At preset times (0, 1, 2, 3, 4, 6, 8, 10 and 24 h), a sample of 100 μl of each of the solutions was taken and diluted in series 10 times in sterile water. An aliquot of 30 μl was then spread on Sabouraud gelose dishes (Biomerieux) in order to count the colonies. When the number of colonies, as estimated, was less than 1000 yeasts/ml, a sample of 30 μl was taken directly from the test solution and spread on Sabouraud gelose dishes (Biomerieux) with no prior dilution. The dishes were incubated for 24 to 48 hours at 35° C.

An Amphotericin B control test (concentration corresponding to 1 time and 16 times MIC) was made in parallel following the protocol described by Klepser et al. (Antimicrob Agents Chemother June 1997, 41(6):1392-1395, “Antiiifungal pharmacodynamic characteristics of fluconazole and Amphotericin B tested against Candida albicans”).

The minimum detection threshold of the number of yeasts/ml was determined by preparing a suspension of Candida albicans yeast in sterile water with the pEM35 or pEM51 peptide then adjustment to 0.5 Mc Farland turbidity standard (concentration 1.10⁶-5.10⁶ yeasts/ml). Dilutions in sterile water were made to obtain 3 suspensions having respective concentrations of 100, 50 and 30 yeasts/ml. 30 μl of each suspension were taken and spread on Sabouraud gelose dishes (Biomerieux) for colony counting. The dishes were incubated for 24 to 48 hours at 35° C.

The values counted (log₁₀ yeasts/ml) were entered into a pre-set time scale for each of the tested concentrations of the pEM35 and pEM51 peptides.

1. A peptide derived from heliomycin by substituting one or more amino acids, comprising peptides meeting formula (I):
X₁ X₂ X₃ X₄ X₅ X₆ C₇ X₈ X₉ X₁₀ X₁₁ X₁₂ X₁₃ X₁₄ X₁₅ X₁₆ X₁₇ C₁₈ X₁₉ X₂₀ X₂₁ C₂₂ X₂₃ X₂₄ X₂₅ X₂₆ X₂₇ X₂₈ X₂₉ X₃₀ X₃₁ C₃₂ X₃₃ X₃₄ X₃₅ X₃₆ X₃₇ X₃₈ X₃₉ C₄₀ X₄₁ C₄₂ X₄₃ X₄₄ (I)
in which:
X₁, X₁₇, X₂₁, X₄₂, are acidic amino acids,
X₁₆, X₄₄ are small polar amino acids,
X₁₉ is a large polar amino acid,
X₃₆ is a small or scarcely hydrophobic amino acid,
X₃₈ is a scarcely hydrophobic or small amino acid, said substitutions being such that:
at least one of X₁, X₁₇, X₂₁, X₄₃, is a basic or polar, advantageously a large polar, amino acid and/or
at least one of the amino acids X₁₆, X₄₄ is a basic amino acid or a large polar amino acid, and/or
X₁₉ is a basic amino acid, and/or
at least one of the amino acids X₃₆, X₃₈ is a strongly hydrophobic amino acid,
and in which other amino acids (X) have the following meanings:
X₁₃, X₃₇, X₃₉ represent large polar amino acids,
X₅, X₁₅, X₃₄, represent small polar amino acids,
X₂, X₂₃, X₂₄, X₂₅, X₂₈, X₃₁, represent basic amino acids,
X₃, X₄, X₈, X₁₂, represent hydrophobic amino acids,
X₉, X₁₆, X₂₇, X₃₅, X₄₁, represent aromatic hydrophobic amino acids,
X₅, X₁₀, X₁₁, X₂₀, X₂₆, X₂₉, X₃₀, X₃₃, represent small amino acids,
C₇, C₁₈, C₂₂, C₃₂, C₄₀, C₄₂, represent cysteines.

2. The peptide according to claim 1, wherrein at least one of X₁, X₁₇, X₄₃, is a basic or polar, advantageously a large polar, amino acid and X₂₁ is an acidic amino acid.

3. The peptide according to claim 1, wherein at least one of X₃₆ and X₃₈ is a non-aromatic strongly hydrophobic amino acid.

4. The peptide according to claim 1, wherein X₁₇ is asparagine or arginine, X₄₃ is glutamic aicd and in which:
X₃₆ is leucine or isoleucine, and/or
X₁₉ is arginine, and/or
X₁₆ is arginine.

5. The peptide according to claim 1, wherein X₁₇ is aspartic acid, X₄₃ is glutamic acid and in which:
X₃₆ is leucine or isoleucine, and/or
X₁₉ is arginine, and/or
X₁₆ is arginine.

6. The peptide according to claim 1, wherein is X₄₃ is glutamine, X₁₇ is asparagine and in which:
X₃₆ is leucine or isoleucine, and/or
X₁₉ is arginine.

7. The peptide according to claim 1, wherein X₄₃ is glutamine and X₁₇ is aspartic acid.

8. The peptide according to claim 1, wherein X₄₃ is glutamine, X₁₇ is aspartic acid and in which:
X₁ is asparagine, and/or
X₃₆ is leucine or isoleucine.

9. The peptide according to claim 1, wherein the basic amino acids are selected from the group consisting of arginine, lysine and histidine.

10. The peptide according to claim 1, wherein the hydrophobic amino acids are:
non-aromatic and selected from the group consisting of methionine, valine, leucine, isoleucine with the proviso that leucine and isoleucine are strongly hydrophobic amino acids and that methionine and valine are scarcely hydrophobic amino acids, or
aromatic and selected from the group consisting of phenyl-alanine, tyrosine and tryptophan.

11. The peptide according to claim 1, wherein the acidic amino acids are selected from the group consisting of aspartic acid and glutamic acid.

12. The peptide according to claim 1, wherein the large polar amino acids are selected from the group consisting of glutamine and asparagine.

13. The peptide according to claim 1, wherein the small polar amino acids are selected from the group consisting of serine and threonine.

14. The peptide according to claim 1, wherein the small acids are selected from the group consisting of glycine and alanine.

15. The peptide according to claim 1, comprising at least one of the following sequences:

| | |
|---|---|
| Helio: | DKLIGSCVWGAVNYTSDCNGECKRRGYKGGHCGSFANVNCWCET |
| Ard1: | DKLIGSCVWGAVNYTSNCNAECKRRGYKGGHCGSFANVNCWCET |
| pEM37: | NKLIGSCVWGAVNYTSDCNGECKRRGYKGGHCGSFANVNCWCET |
| pEM38: | DKLIGTCVWGAVNYTSDCNGECKRRGYKGGHCGSFANVNCWCET |
| pEM43: | DKLIGSCVWGAVNYTTDCNGECKRRGYKGGHCGSFANVNCWCET |
| pEM42: | DKLIGSCVWGAVNYTRDCNGECKRRGYKGGHCGSFANVNCWCET |
| pEM44: | DKLIGSCVWGAVNYTSDCRGECKRRGYKGGHCGSFANVNCWCET |
| pEM22: | DKLIGSCVWGAVNYTSDCNGECKRRGYKGGHCGSFINVNCWCET |
| pEM23: | DKLIGSCVWGAVNYTSDCNGECKRRGYKGGHCGSFANINCWCET |
| pEM25: | DKLIGSCVWGAVNYTSDCNGECKRRGYKGGHCGSFLNVNCWCET |
| pEM24: | DKLIGSCVWGAVNYTSDCNGECKRRGYKGGHCGSFLNINCWCET |
| pEM7: | DKLIGSCVWGAVNYTSDCNGECKRRGYKGGHCGSPANVNCWCER |
| pEM21: | DKLIGSCVWGAVNYTSDCNGECKRRGYKGGHCGSFANVNCWCQT |
| pEM39: | NKLIGSCVWGAVNYTSDCNGECKRRGYKGGHCGSFANVNCWCQT |
| pEM61: | NKLIGSCVWGAVNYTSDCNGECKRRGYKGGHCGSFLNVNCWCQT |
| pEM62: | NKLIGSCVWGAVNYTSDCNGECKRRGYKGGHCGSFINVNCWCQT |

16. The peptide according to claim 1, comprising at least one of the following sequences:
Ard1: DKLIGSCVWGAVNYTSNCNACKRRGYKGGRCGSFEPTNCWCET
pEM40: NKLIGSCVWGAVNYTSNCGCMGYKGGHCGSFWNCWCET

| | |
|---|---|
| pEM50: | DKLIGSCVWGAVNYTRNCNAECKRRGYKGGHCGSFANVNCWCET |
| pEM56: | DKLIGSCVWLAVNYTSNCNAECKRRGYKGGHCGSFANVNCWCET |
| pEM52: | DKLIGSCVWGAVNYTSRCNAECKRRGYKGGHCGSFANVNCWCET |
| pEM51: | DKLIGSCVWGAVNYTSNCRAECKRRGYKGGHCGSFANVNCWCET |
| pEM32: | DKLIGSCVWGAVNYTSNCNAECKRRGYKGGHCGSFINVNCWCET |
| pEM33: | DKLIGSCVWGAVNYTSNCNAECKRRGYKGGHCGSFANINCWCET |
| pEM34: | DKLIGSCVWGAVNYTSNCNAECKRRGYKGGHCGSFLNINCWCET |
| pEM35: | DKLIGSCVWGAVNYTSNCNAECKRRGYKGGHCGSFLNVNCWCET |
| pEM31: | DKLIGSCVWGAVNYTSNCNAECKRRGYKGGHCGSFANVNCWCQT |
| pEM30: | DKLIGSCVWGAVNYTSNCNAECKRRGYKGGHCGSFANVNCWCER |
| pEM46: | DKLIGSCVWGAVNYTSNCNAECKRRGYKGGHCGSFLNVNCWCQT |
| pEM47: | DKLIGSCVWGAVNYTSNCNAECKRRGYKGGHCGSFINVNCWCQT |
| pEM48: | DKLIGSCVWGAVNYTSNCNAECKRRGYKGGHCGSFLNVNCWCER |
| pEM49: | DKLIGSCVWGAVNYTSNCNAECKRRGYKGGHCGSFINVNCWCER |
| pEM54: | DKLIGSCVWLAVNYTSNCNAECKRRGYKGGHCGSFLNVNCWCET |
| pEM57: | DKLIGSCVWLAVNYTSNCNAECKRRGYKGGHCGSFANVNCWCQT |
| pEM55: | DKLIGSCVWLAVNYTSNCNAECKRRGYKGGHCGSFLNVNCWCQT |

17. An antifungal and/or antibacterial composition comprising a therapeutically effective amount of at least one peptide according to claim 1, and a pharmaceutically acceptable vehicle.

18. A method of treating fungal or bacterial infection comprising administering a therapeutically effective amount of the composition of claim 17 to a mammal.

19. A method of treating fungal or bacterial infection comprising administering a therapeutically effective amount of the composition of claim 17 to a plant.

20. A peptide derived from heliomicine, comprising an amino acid sequence corresponding to a heliomicine sequence in which hydrophobic and charged regions have one or several mutations and the peptide containing at least one of the following sequences:
Ard1:
DKLIGSCVWGAVNYTSNCNAECKRRGYKGGHCGSFANVNCWCET
pEM37:
NKLIGSCVWGAVNYTSDCNGECKRRGYKGGHCGSFANVNCWCET
pEM43:
DKLIGSCVWGAVNYTTDCNGECKRRGYKGGHCGSFANVNCWCET
pEM42:
DKLIGSCVWGAVNYTRDCNGECKRRGYKGGHCGSFANVNCWCET
pEM44:
DKLIGSCVWGAVNYTSDCRGECKRRGYKGGHCGSFANVNCWCET
pEM22:
DKLIGSCVWGAVNYTSDCNGECKRRGYKGGHCGSFINVNCWCET
pEM23:
DKLIGSCVWGAVNYTSDCNGECKRRGYKGGHCGSFANINCWCET
pEM25:
DKLTGSCVWGAVNYTSDCNGECKRRGYKGGHCGSFLNVNCWCET
pEM24:
DKLIGSCVWGAVNYTSDCNGECKRRGYKGGHCGSFLNINCWCET
pEM7:
DKLIGSCVWGAVNYTSDCNGECKRRGYKGGHCGSFANVNCWCER
pEM21:
DKLIGSCVWGAVNYTSDCNGECKRRGYKGGHCGSFANVNCWCQT
pEM39:
NKLIGSCVWGAVNYTSDCNGECKRRGYKGGHCGSFANVNCWCQT
pEM61:
NKLTGSCVWGAVNYTSDCNGECKRRGYKGGHCGSFLNVNCWCQT
pEM62:
NKLIGSCVWGAVNYTSDCNGECKRRGYKGGHCGSFINVNCWCQT.

21. A peptide derived from heliomicine, comprising an amino acid sequence corresponding to a heliomicine sequence in which hydrophobic and charged regions have one or several mutations and the peptide contains at least one of the following sequences:
Ard1:
DKLIGSCVWGAVNYTSNCNAECKRRGYKGGHCGSFANVNCWCET
pEM40:
NKLIGSCVWGAVNYTSNCNAECKRRGYKGGHCGSFANVNCWCET
pEM50:
DKLIGSCVWGAVNYTRNCNAECKRRGYKGGHCGSFANVNCWCET
pEM56:
DKLIGSCVWLAVNYTSNCNAECKRRGYKGGHCGSFANVNCWCET
pEM52:
DKLIGSCVWGAVNYTSRCNAECKRRGYKGGHCGSFANVNCWCET
pEM51:
DKLIGSCVWGAVNYTSNCRAECKRRGYKGGHCGSFANVNCWCET
pEM32:
DKLIGSCVWGAVNYTSNCNAECKRRGYKGGHCGSFINVNCWCET
pEM33:
DKLIGSCVWGAVNYTSNCNAECKRRGYKGGHCGSFANINCWCET
pEM34:
DKLIGSCVWGAVNYTSNCNAECKRRGYKGGHCGSFLNINCWCET
pEM35:
DKLIGSCVWGAVNYTSNCNAECKRRGYKGGHCGSFLNVNCWCET
pEM31:
DKLIGSCVWGAVNYTSNCNAECKRRGYKGGHCGSFANVNCWCQT
pEM30:
DKLIGSCVWGAVNYTSNCNAECKRRGYKGGHCGSFANVNCWCER
pEM46:
DKLIGSCVWGAVNYTSNCNAECKRRGYKGGHCGSFLNVNCWCQT
pEM47:
DKLIGSCVWGAVNYTSNCNAECKRRGYKGGHCGSFINVNCWCQT
pEM48:
DKLIGSCVWGAVNYTSNCNAECKRRGYKGGHCGSFLNVNCWCER
pEM49:
DKLIGSCVWGAVNYTSNCNAECKRRGYKGGHCGSFINVNCWCER
pEM54:
DKLIGSCVWLAVNYTSNCNAECKRRGYKGGHCGSFLNVNCWCET
pEM57:
DKLIGSCVWLAVNYTSNCNAECKRRGYKGGHCGSFANVNCWCQT
pEM55:
DKLIGSCVWLAVNYTSNCNAECKRRGYKGGHCGSFLNVNCWCQT.

22. An antifungal and/or antibacterial composition comprising at least one peptide according to claim 20 and a pharmaceutically acceptable vehicle.

23. An antifungal and/or antibacterial composition comprising at least one peptide according to claim 21 and a pharmaceutically acceptable vehicle.

24. A method of treating fungal or bacterial infection comprising administering a therapeutically effective amount of the composition of claim 22 to a mammal.

25. A method of treating fungal or bacterial infection comprising administering a therapeutically effective amount of the composition of claim 23 to a mammal.

26. A method of treating fungal or bacterial infection comprising administering a therapeutically effective amount of the composition of claim 22 to a plant.

27. A method of treating fungal or bacterial infection comprising administering a therapeutically effective amount of the composition of claim 23 to a plant.

28. A nucleic acid sequence, characterized in that it is able to express a peptide according to any of claims 20 or 21.

29. An expression vector comprising a nucleic acid sequence according to claim 28.

30. A plant cell comprising a nucleic acid sequence according to claim 28.

31. A disease resistant plant comprising a nucleic acid sequence according to claim 28.


SEQUENCE LISTING (97 sequences)

SEQ ID NO: 1 - protein, 44 aa, Artificial Sequence
  Description of Artificial Sequence Synthetic heliomicine
DKLIGSCVWGAVNYTSDCNGECKRRGYKGGHCGSFANVNCWCET

SEQ ID NO: 2 - protein, 44 aa, Artificial Sequence
  Description of Artificial Sequence Synthetic Ard1, peptide homologue to heliomicine
DKLIGSCVWGAVNYTSNCNAECKRRGYKGGHCGSFANVNCWCET

SEQ ID NO: 3 - protein, 44 aa, Artificial Sequence
  Description of Artificial Sequence Synthetic pEM37 peptide derived from heliomicine
NKLIGSCVWGAVNYTSDCNGECKRRGYKGGHCGSFANVNCWCET

SEQ ID NO: 4 - protein, 44 aa, Artificial Sequence
  Description of Artificial Sequence Synthetic pEM38 peptide derived from heliomicine
DKLIGTCVWGAVNYTSDCNGECKRRGYKGGHCGSFANVNCWCET

SEQ ID NO: 5 - protein, 44 aa, Artificial Sequence
  Description of Artificial Sequence Synthetic pEM43 peptide derived from heliomicine
DKLIGSCVWGAVNYTTDCNGECKRRGYKGGHCGSFANVNCWCET

SEQ ID NO: 6 - protein, 44 aa, Artificial Sequence
  Description of Artificial Sequence Synthetic pEM42 peptide derived from heliomicine
DKLIGSCVWGAVNYTRDCNGECKRRGYKGGHCGSFANVNCWCET

SEQ ID NO: 7 - protein, 44 aa, Artificial Sequence
  Description of Artificial Sequence Synthetic pEM44 peptide derived from heliomicine
DKLIGSCVWGAVNYTSDCRGECKRRGYKGGHCGSFANVNCWCET

SEQ ID NO: 8 - protein, 44 aa, Artificial Sequence
  Description of Artificial Sequence Synthetic pEM22 peptide derived from heliomicine
DKLIGSCVWGAVNYTSDCNGECKRRGYKGGHCGSFINVNCWCET

SEQ ID NO: 9 - protein, 44 aa, Artificial Sequence; identical to SEQ ID NO: 1
  Description of Artificial Sequence Synthetic pEM23 peptide derived from heliomicine

SEQ ID NO: 10 - protein, 44 aa, Artificial Sequence
  Description of Artificial Sequence Synthetic pEM25 peptide derived from heliomicine
DKLIGSCVWGAVNYTSDCNGECKRRGYKGGHCGSFLNVNCWCET

SEQ ID NO: 11 - protein, 44 aa, Artificial Sequence
  Description of Artificial Sequence Synthetic pEM24 peptide derived from heliomicine
DKLIGSCVWGAVNYTSDCNGECKRRGYKGGHCGSFLNINCWCET

SEQ ID NO: 12 - protein, 44 aa, Artificial Sequence
  Description of Artificial Sequence Synthetic pEM7 peptide derived from heliomicine
DKLIGSCVWGAVNYTSDCNGECKRRGYKGGHCGSFANVNCWCER

SEQ ID NO: 13 - protein, 44 aa, Artificial Sequence
  Description of Artificial Sequence Synthetic pEM21 peptide derived from heliomicine
DKLIGSCVWGAVNYTSDCNGECKRRGYKGGHCGSFANVNCWCQT

SEQ ID NO: 14 - protein, 44 aa, Artificial Sequence
  Description of Artificial Sequence Synthetic pEM39 peptide derived from heliomicine
NKLIGSCVWGAVNYTSDCNGECKRRGYKGGHCGSFANVNCWCQT

SEQ ID NO: 15 - protein, 44 aa, Artificial Sequence
  Description of Artificial Sequence Synthetic pEM61 peptide derived from heliomicine
NKLIGSCVWGAVNYTSDCNGECKRRGYKGGHCGSFLNVNCWCQT

SEQ ID NO: 16 - protein, 44 aa, Artificial Sequence
  Description of Artificial Sequence Synthetic pEM62 peptide derived from heliomicine
NKLIGSCVWGAVNYTSDCNGECKRRGYKGGHCGSFINVNCWCQT

SEQ ID NO: 17 - protein, 44 aa, Artificial Sequence
  Description of Artificial Sequence Synthetic pEM4 peptide derived from heliomicine
NKLIGSCVWGAVNYTSNCNAECKRRGYKGGHCGSFANVNCWCET

SEQ ID NO: 18 - protein, 44 aa, Artificial Sequence
  Description of Artificial Sequence Synthetic pEM5 peptide derived from heliomicine
DKLIGSCVWGAVNYTRNCNAECKRRGYKGGHCGSFANVNCWCET

SEQ ID NO: 19 - protein, 44 aa, Artificial Sequence
  Description of Artificial Sequence Synthetic pEM5 peptide derived from heliomicine
DKLIGSCVWLAVNYTSNCNAECKRRGYKGGHCGSFANVNCWCET

SEQ ID NO: 20 - protein, 44 aa, Artificial Sequence
  Description of Artificial Sequence Synthetic pEM5 peptide derived from heliomicine
DKLIGSCVWGAVNYTSRCNAECKRRGYKGGHCGSFANVNCWCET

SEQ ID NO: 21 - protein, 44 aa, Artificial Sequence
  Description of Artificial Sequence Synthetic pEM5 peptide derived from heliomicine
DKLIGSCVWGAVNYTSNCRAECKRRGYKGGHCGSFANVNCWCET

SEQ ID NO: 22 - protein, 44 aa, Artificial Sequence
  Description of Artificial Sequence Synthetic pEM3 peptide derived from heliomicine
DKLIGSCVWGAVNYTSNCNAECKRRGYKGGHCGSFINVNCWCET

SEQ ID NO: 23 - protein, 44 aa, Artificial Sequence
  Description of Artificial Sequence Synthetic pEM3 peptide derived from heliomicine
DKLIGSCVWGAVNYTSNCNAECKRRGYKGGHCGSFANINCWCET

SEQ ID NO: 24 - protein, 44 aa, Artificial Sequence
  Description of Artificial Sequence Synthetic pEM3 peptide derived from heliomicine
DKLIGSCVWGAVNYTSNCNAECKRRGYKGGHCGSFLNINCWCET

SEQ ID NO: 25 - protein, 44 aa, Artificial Sequence
  Description of Artificial Sequence Synthetic pEM3 peptide derived from heliomicine
DKLIGSCVWGAVNYTSNCNAECKRRGYKGGHCGSFLNVNCWCET

SEQ ID NO: 26 - protein, 44 aa, Artificial Sequence
  Description of Artificial Sequence Synthetic pEM3 peptide derived from heliomicine
DKLIGSCVWGAVNYTSNCNAECKRRGYKGGHCGSFANVNCWCQT

SEQ ID NO: 27 - protein, 44 aa, Artificial Sequence
  Description of Artificial Sequence Synthetic pEM3 peptide derived from heliomicine
DKLIGSCVWGAVNYTSNCNAECKRRGYKGGHCGSFANVNCWCER

SEQ ID NO: 28 - protein, 44 aa, Artificial Sequence
  Description of Artificial Sequence Synthetic pEM4 peptide derived from heliomicine
DKLIGSCVWGAVNYTSNCNAECKRRGYKGGHCGSFLNVNCWCQT

SEQ ID NO: 29 - protein, 44 aa, Artificial Sequence
  Description of Artificial Sequence Synthetic pEM4 peptide derived from heliomicine
DKLIGSCVWGAVNYTSNCNAECKRRGYKGGHCGSFINVNCWCQT

SEQ ID NO: 30 - protein, 44 aa, Artificial Sequence
  Description of Artificial Sequence Synthetic pEM4 peptide derived from heliomicine
DKLIGSCVWGAVNYTSNCNAECKRRGYKGGHCGSFLNVNCWCER

SEQ ID NO: 31 - protein, 44 aa, Artificial Sequence
  Description of Artificial Sequence Synthetic pEM4 peptide derived from heliomicine
DKLIGSCVWGAVNYTSNCNAECKRRGYKGGHCGSFINVNCWCER

SEQ ID NO: 32 - protein, 44 aa, Artificial Sequence
  Description of Artificial Sequence Synthetic pEM5 peptide derived from heliomicine
DKLIGSCVWLAVNYTSNCNAECKRRGYKGGHCGSFLNVNCWCET

SEQ ID NO: 33 - protein, 44 aa, Artificial Sequence
  Description of Artificial Sequence Synthetic pEM5 peptide derived from heliomicine
DKLIGSCVWLAVNYTSNCNAECKRRGYKGGHCGSFANVNCWCQT

SEQ ID NO: 34 - protein, 44 aa, Artificial Sequence
  Description of Artificial Sequence Synthetic pEM5 peptide derived from heliomicine
DKLIGSCVWLAVNYTSNCNAECKRRGYKGGHCGSFLNVNCWCQT

SEQ ID NO: 35 - protein, 44 aa, Artificial Sequence; identical to SEQ ID NO: 1
  Description of Artificial Sequence Synthetic pEM3 peptide derived from heliomicine

SEQ ID NO: 36 - protein, 44 aa, Artificial Sequence; identical to SEQ ID NO: 4
  Description of Artificial Sequence Synthetic pEM3 peptide derived from heliomicine

SEQ ID NO: 37 - protein, 44 aa, Artificial Sequence
  Description of Artificial Sequence Synthetic pEM4 peptide derived from heliomicine
DKLIGSCVWGAVRYTSDCNGECKRRGYKGGHCGSFANVNCWCET

SEQ ID NO: 38 - protein, 44 aa, Artificial Sequence; identical to SEQ ID NO: 6
  Description of Artificial Sequence Synthetic pEM4 peptide derived from heliomicine

SEQ ID NO: 39 - protein, 44 aa, Artificial Sequence; identical to SEQ ID NO: 7
  Description of Artificial Sequence Synthetic pEM4 peptide derived from heliomicine

SEQ ID NO: 40 - protein, 44 aa, Artificial Sequence; identical to SEQ ID NO: 8
  Description of Artificial Sequence Synthetic pEM2 peptide derived from heliomicine

SEQ ID NO: 41 - protein, 44 aa, Artificial Sequence
  Description of Artificial Sequence Synthetic pEM2 peptide derived from heliomicine
DKLIGSCVWGAVNYTSDCNGECKRRGYKGGHCGSFANINCWCET

SEQ ID NO: 42 - protein, 44 aa, Artificial Sequence; identical to SEQ ID NO: 10
  Description of Artificial Sequence Synthetic pEM2 peptide derived from heliomicine

SEQ ID NO: 43 - protein, 44 aa, Artificial Sequence; identical to SEQ ID NO: 11
  Description of Artificial Sequence Synthetic pEM2 peptide derived from heliomicine

SEQ ID NO: 44 - protein, 44 aa, Artificial Sequence; identical to SEQ ID NO: 12
  Description of Artificial Sequence Synthetic pEM peptide derived from heliomicine

SEQ ID NO: 45 - protein, 44 aa, Artificial Sequence; identical to SEQ ID NO: 13
  Description of Artificial Sequence Synthetic pEM2 peptide derived from heliomicine

SEQ ID NO: 46 - protein, 44 aa, Artificial Sequence; identical to SEQ ID NO: 13
  Description of Artificial Sequence Synthetic pEM3 peptide derived from heliomicine

SEQ ID NO: 47 - protein, 44 aa, Artificial Sequence
  Description of Artificial Sequence Synthetic pEM6 peptide derived from heliomicine
NKLIGSCVWGAVNYTSDCNGECKRRGYKGGHCGSFANLNCWCQT

SEQ ID NO: 48 - protein, 44 aa, Artificial Sequence
  Description of Artificial Sequence Synthetic pEM6 peptide derived from heliomicine
NKLIGSCVWGAVNYTSDCNGECKRRGYKGGHCGSFANINCWCQT

SEQ ID NO: 49 - protein, 44 aa, Artificial Sequence; identical to SEQ ID NO: 17
  Description of Artificial Sequence Synthetic pEM5 peptide derived from heliomicine

SEQ ID NO: 50 - protein, 44 aa, Artificial Sequence; identical to SEQ ID NO: 18
  Description of Artificial Sequence Synthetic pEM5 peptide derived from heliomicine

SEQ ID NO: 51 - protein, 44 aa, Artificial Sequence; identical to SEQ ID NO: 19
  Description of Artificial Sequence Synthetic pEM5 peptide derived from heliomicine

SEQ ID NO: 52 - protein, 44 aa, Artificial Sequence; identical to SEQ ID NO: 20
  Description of Artificial Sequence Synthetic pEM5 peptide derived from heliomicine

SEQ ID NO: 53 - protein, 44 aa, Artificial Sequence; identical to SEQ ID NO: 21
  Description of Artificial Sequence Synthetic pEM5 peptide derived from heliomicine

SEQ ID NO: 54 - protein, 44 aa, Artificial Sequence; identical to SEQ ID NO: 22
  Description of Artificial Sequence Synthetic pEM3 peptide derived from heliomicine

SEQ ID NO: 55 - protein, 44 aa, Artificial Sequence; identical to SEQ ID NO: 23
  Description of Artificial Sequence Synthetic pEM3 peptide derived from heliomicine

SEQ ID NO: 56 - protein, 44 aa, Artificial Sequence; identical to SEQ ID NO: 24
  Description of Artificial Sequence Synthetic pEM3 peptide derived from heliomicine

SEQ ID NO: 57 - protein, 44 aa, Artificial Sequence; identical to SEQ ID NO: 25
  Description of Artificial Sequence Synthetic pEM3 peptide derived from heliomicine

SEQ ID NO: 58 - protein, 44 aa, Artificial Sequence; identical to SEQ ID NO: 26
  Description of Artificial Sequence Synthetic pEM3 peptide derived from heliomicine

SEQ ID NO: 59 - protein, 44 aa, Artificial Sequence; identical to SEQ ID NO: 27
  Description of Artificial Sequence Synthetic pEM3 peptide derived from heliomicine

SEQ ID NO: 60 - protein, 44 aa, Artificial Sequence; identical to SEQ ID NO: 28
  Description of Artificial Sequence Synthetic pEM4 peptide derived from heliomicine

SEQ ID NO: 61 - protein, 44 aa, Artificial Sequence; identical to SEQ ID NO: 29
  Description of Artificial Sequence Synthetic pEM4 peptide derived from heliomicine

SEQ ID NO: 62 - protein, 44 aa, Artificial Sequence; identical to SEQ ID NO: 30
  Description of Artificial Sequence Synthetic pEM4 peptide derived from heliomicine

SEQ ID NO: 63 - protein, 44 aa, Artificial Sequence; identical to SEQ ID NO: 31
  Description of Artificial Sequence Synthetic pEM4 peptide derived from heliomicine

SEQ ID NO: 64 - protein, 44 aa, Artificial Sequence; identical to SEQ ID NO: 32
  Description of Artificial Sequence Synthetic pEM5 peptide derived from heliomicine

SEQ ID NO: 65 - protein, 44 aa, Artificial Sequence; identical to SEQ ID NO: 33
  Description of Artificial Sequence Synthetic pEM5 peptide derived from heliomicine

SEQ ID NO: 66 - protein, 44 aa, Artificial Sequence; identical to SEQ ID NO: 33
  Description of Artificial Sequence Synthetic pEM5 peptide derived from heliomicine

SEQ ID NO: 67 - DNA, 26 bp, Artificial Sequence
  Description of Artificial Sequence Synthetic oligonucleotide EM72
gtaaatgcatgtatactaaactcaca

SEQ ID NO: 68 - DNA, 55 bp, Artificial Sequence
  Description of Artificial Sequence Synthetic oligonucleotide EM89
ttttttccgcggcgcttgcactcggcgttgcagttactagtgtagttgacggcgc

SEQ ID NO: 69 - DNA, 47 bp, Artificial Sequence
  Description of Artificial Sequence Synthetic oligonucleotide EM89 complementary strand
cgccgcgaacgtgagccgcaacgtcaatgatcacatcaactgccgcg

SEQ ID NO: 70 - protein, 15 aa, Artificial Sequence
  Description of Artificial Sequence Synthetic mutated peptide analogous to Ard1
RRKCEANCNSTYNVA

SEQ ID NO: 71 - DNA, 20 bp, Artificial Sequence
  Description of Artificial Sequence Synthetic oligonucleotide EM25
gatccactcgagtgctagcg

SEQ ID NO: 72 - DNA, 20 bp, Artificial Sequence
  Description of Artificial Sequence Synthetic oligonucleotide EM26
tcgacgctagcactcgagtg

SEQ ID NO: 73 - DNA, 42 bp, Artificial Sequence
  Description of Artificial Sequence Synthetic oligonucleotide EM119
gatccttcattaacgttaactgttggtgtgaaacctgatagg

SEQ ID NO: 74 - protein, 11 aa, Artificial Sequence
  Description of Artificial Sequence Synthetic mutated peptide (oligonucleotides EM119 and EM120)
SFINVNCWCET

SEQ ID NO: 75 - DNA, 42 bp, Artificial Sequence
  Description of Artificial Sequence Synthetic oligonucleotide EM120
tcgacctatcaggtttcacaccaacagttaacgttaatgaag

SEQ ID NO: 76 - DNA, 42 bp, Artificial Sequence
  Description of Artificial Sequence Synthetic oligonucleotide EM127
gatccttcttgaacattaactgttggtgtgaaacctgatagg

SEQ ID NO: 77 - protein, 11 aa, Artificial Sequence
  Description of Artificial Sequence Synthetic mutated peptide (oligonucleotides EM127 and EM128)
SFLNVNCWCET

SEQ ID NO: 78 - DNA, 42 bp, Artificial Sequence
  Description of Artificial Sequence Synthetic oligonucleotide EM128
tcgacctatcaggtttcacaccaacagttaatgttcaagaag

SEQ ID NO: 79 - DNA, 42 bp, Artificial Sequence
  Description of Artificial Sequence Synthetic oligonucleotide EM117
gatccttcgctaacgttaactgttggtgtcaaacctgatagg

SEQ ID NO: 80 - protein, 11 aa, Artificial Sequence
  Description of Artificial Sequence Synthetic mutated peptide (oligonucleotides EM117 and EM118)
SFANVNCWCQT

SEQ ID NO: 81 - DNA, 42 bp, Artificial Sequence
  Description of Artificial Sequence Synthetic oligonucleotide EM118
tcgacctatcaggtttgacaccaacagttaacgttagcgaag

SEQ ID NO: 82 - DNA, 42 bp, Artificial Sequence
  Description of Artificial Sequence Synthetic oligonucleotide EM129
gatccttcttgaacgttaactgttggtgtgaaacctgatagg

SEQ ID NO: 83 - protein, 11 aa, Artificial Sequence
  Description of Artificial Sequence Synthetic mutated peptide (oligonucleotides EM129 and EM130)
SFLNVNCWCET

SEQ ID NO: 84 - DNA, 42 bp, Artificial Sequence
  Description of Artificial Sequence Synthetic oligonucleotide EM130
tcgacctatcaggtttcacaccaacagttaacgttcaagaag

SEQ ID NO: 85 - DNA, 42 bp, Artificial Sequence
  Description of Artificial Sequence Synthetic oligonucleotide EM135
gatccttcgctaacgttaactgttggtgtgaaagatgatagg

SEQ ID NO: 86 - protein, 11 aa, Artificial Sequence
  Description of Artificial Sequence Synthetic mutated peptide (oligonucleotides EM135 and EM136)
SFANVNCWCER

SEQ ID NO: 87 - DNA, 42 bp, Artificial Sequence
  Description of Artificial Sequence Synthetic oligonucleotide EM136
tcgacctatcatctttcacaccaacagttaacgttagcgaag

SEQ ID NO: 88 - DNA, 42 bp, Artificial Sequence
  Description of Artificial Sequence Synthetic oligonucleotide EM158
gatccttcttgaacgttaactgttggtgtcaaacctgatagg

SEQ ID NO: 89 - protein, 11 aa, Artificial Sequence
  Description of Artificial Sequence Synthetic mutated peptide (oligonucleotides EM158 and EM159)
SFLNVNCWCQT

SEQ ID NO: 90 - DNA, 42 bp, Artificial Sequence
  Description of Artificial Sequence Synthetic oligonucleotide EM159
tcgacctatcaggtttgacaccaacagttaacgttcaagaag

SEQ ID NO: 91 - DNA, 42 bp, Artificial Sequence
  Description of Artificial Sequence Synthteic oligonucleotide EM162
gatccttcttgaacgttaactgttggtgtgaaagatgatagg

SEQ ID NO: 92 - protein, 11 aa, Artificial Sequence
  Description of Artificial Sequence Synthetic mutated peptide (oligonucleotides EM162 and EM163)
SFLNVNCWCER

SEQ ID NO: 93 - DNA, 42 bp, Artificial Sequence
  Description of Artificial Sequence Synthetic oligonucleotide EM163
tcgacctatcatctttcacaccaacagttaacgttcaagaag

SEQ ID NO: 94 - DNA, 22 bp, Artificial Sequence
  Description of Artificial Sequence Synthetic oligonucleotide EM53
cctggcaattccttaccttcca

SEQ ID NO: 95 - DNA, 39 bp, Artificial Sequence
  Description of Artificial Sequence Synthetic oligonucleotide EM137
ttttttaagcttggataaaagaaacaagttgattggcag

SEQ ID NO: 96 - protein, 10 aa, Artificial Sequence
  Description of Artificial Sequence Synthetic mutated peptide (oligonucleotides EM53 and EM137)
SLDKRNKLIG

SEQ ID NO: 97 - protein, 44 aa, Artificial Sequence
  Description of Artificial Sequence Synthetic peptide
XXXXXXCXXXXXXXXXXCXXXCXXXXXXXXXCXXXXXXXCXCXX